(19) United States
(12) Patent Application Publication
(10) Pub. No.: US 2006/0051430 A1
(43) Pub. Date: Mar. 9, 2006

(54) Silver dihydrogen citrate compositions

(76) Inventors: Andrew B. Arata, Lake City, FL (US); Andrea Preuss, Basel (CH)

Correspondence Address: McDERMOTT WILL & EMERY LLP, Suite 700, 4370 La Jolla Village Drive, San Diego, CA, 92122, US

(21) Appl. No.: 10/936,465

(22) Filed: Sep. 7, 2004

(51) Int. Cl. A61K 31/28; A61K 33/38

(52) U.S. Cl. 424/618; 514/495

(57) ABSTRACT

Personal care and home care compositions are disclosed, which include silver dihydrogen citrate. The inventive compositions advantageously take the form of suspensions, pastes, liquids and gels. The inventive compositions also optionally comprise additional ingredients, and are therefore suitable for a wide variety of personal, home and industrial care purposes.

### FIELD OF THE INVENTION

The present invention relates to personal care and home care products containing antimicrobial compositions and, more specifically, to silver dihydrogen citrate-containing antimicrobial compositions.

### BACKGROUND OF THE INVENTION

Consumers use a variety of personal care products for the enhancement or maintenance of health and personal appearance. Such products find use, for example, in skin care; as antiperspirants and deodorants; as personal cleansers; in hair care; as oral care products; and as decorative cosmetics. Such products often include antimicrobial agents, which serve to kill or control the growth of undesirable microbes such as bacteria, fungi and viruses. Antimicrobial agents are often employed in such divergent products as deodorant sprays, foot treatments and toothpastes, to name but a few. Antimicrobial agents may be used as preservatives to prevent the growth of bacteria which may contaminate deodorants or cosmetics, for example upon contact with the skin. Alternatively, the antimicrobial effect of such compositions may provide benefit in their end use. For example, antimicrobial agents in foot care products, are often used to treat dermatophyte infections. In tooth care products, antimicrobials are used as anti-caries, anti-gingivitis and anti-periodontitis agents.

Home care products are used to clean or disinfect in homes, hospitals, hotels, motels and offices, and other places where human beings live, work or otherwise carry on the functions of life. In general, home care products confer some benefit on the consumer by acting on the consumer's environment, whether it be at home, work, or in places of public convenience. Antimicrobials are employed in home care products, such as detergents, soaps, bleaches, antiseptics, deodorants and other cleansing agents, to kill microbes or to control their growth. Microbes include bacteria, fungi and viruses. In some instances, the antimicrobial agents may act as preservatives for the home care product. In addition, manufacturers often include antimicrobials in their products in order to confer an antimicrobial benefit through the intended use of the product. For example, a manufacturer may include an antimicrobial agent in a laundry detergent in order to remove detrimental microbes, for example bacteria, viruses and fungi, from clothing. A manufacturer may include an antimicrobial agent in surface washing detergent in order to reduce the viability or number of microbes on a variety of surfaces in the user's environment. Indeed, manufacturers have found antimicrobials to be useful in a vast variety of home care products.

In both personal care and home care products, antimicrobials provide a distinct advantage as preservatives in extending the useful life of the product. Price competition for such products is intense, so manufacturers are continuously called upon to reduce costs, both of manufacturing and loss. In order to reduce the cost of lost product, manufacturers must ensure that the product is protected throughout the entire supply chain. Once a product is manufactured, it is packaged, shipped, and in some cases stored for long periods, before being purchased by consumers. Even after a consumer buys a product, the product may sit unused, and perhaps opened, exposing it to microbial contamination.

Manufacturers must take these considerations into account and must design products to be stable throughout all the stages of the product's life-cycle, including manufacturing and packaging; wholesale and retail; and purchase and eventual use. In the course of a product's life-cycle, it comes into contact with a variety of microbes, including bacteria, viruses, and fungi. In an effort to prevent microbes from degrading the quality of their products, manufacturers can add antimicrobial agents to their products. Indeed, manufacturers have succeeded in greatly enhancing the quality of their products by adding such antimicrobials.

Although a number of antimicrobials have been developed for use personal care and home care products, the diversity of microbes, and their tendency to produce resistant mutants, places constant pressure on manufacturers to produce improved antimicrobials. Ideally, such antimicrobials would combine the properties of low cost and ability to combat a wide variety of microbes. In considering the cost of an antimicrobial agent, not only the cost of initial manufacture, but also the range of concentrations at which it is effective, are important. An improved antimicrobial must be relatively inexpensive to manufacture, and should be active in a range of concentrations that would make its inclusion in the desired product economical. In addition, it would be desirable for such an antimicrobial agent to have a broad spectrum of activity, so as to be effective against a wide variety of microbes. The need for increasingly cost-effective and broad spectrum antimicrobials is ongoing and seemingly endless.

While silver ion compositions for treating water have been known in the art, generally the silver ion species are short-lived once they have been dissolved in water. Thus, it has previously been necessary to prepare solutions containing silver ion species at the point-of-use. While reconstituting an aqueous solution comprising silver ions may have limited potential for some large-scale operations, it is not convenient in the realm of home care, where consumers prefer ready-to-use compositions or liquid compositions that can be diluted in water and used. In applications where silver ions might represent an alternative antimicrobial preservative (for example in shampoos and other water-containing compositions), silver ions have not been previously exploited due in part to this relative instability.

Silver salts, such as silver citrate salts, have also been proposed as antimicrobial dusting agents. However, these dusting agents must be kept dry and are generally not convenient for imparting preservative value to consumer products or for delivering antimicrobial effects to an end user or to the environment of the end user.

Recently, there have been provided silver-ion containing compositions comprising silver ions in complex with citric acid. See, for example, U.S. Pat. No. 6,583,176. However, no personal care or home care products utilizing these compositions has been previously described.

From the foregoing, it is apparent that there is a need for personal care products and home care products having broad scale antimicrobial properties. Such products need to be non-toxic and non-irritating in human use. Ideally, such products should have a long shelf life and be inexpensive. The present invention satisfies these needs and provides related advantages as well.

### SUMMARY OF THE INVENTION

The present invention provides personal care compositions that contain silver dihydrogen citrate in a physiologically acceptable medium. Such silver dihydrogen citrate containing compositions can include additional ingredients, either soluble or non-soluble, such as other antimicrobials, and can also include a detergent or alcohol. The personal care compositions that contain silver dihydrogen citrate and be formulated in a variety of ways, and may include both an aqueous and a non-aqueous, or oil, phase, optionally including an emulsifying agent. Additionally the compositions can include gelling agents or thickening agents.

The invention further provides personal care compositions that contain a water phase, an oil phase and an emulsifier. The water phase contains water and silver dihydrogen citrate. The compositions are emulsions of the following types: water-in-oil, oil-in-water, water-in-oil-in-water, oil-in-water-in-oil, phase inversion temperature, or microemulsions.

The invention further provides methods of using the personal care compositions of the invention. The methods include contacting the inventive personal care compositions with a part of the human body.

The invention further provides home care compositions. The home care compositions contain silver dihydrogen citrate, water, and at least one ingredient other than a detergent or an alcohol. Such silver dihydrogen citrate-containing home care compositions can include additional ingredients, either soluble or non-soluble, such as other antimicrobials, enzymes, bleaches, whiteners, color care agents, fabric softeners, suds suppressors, dispersants, dye transfer inhibitors, chelating agents and aerosol propellants and can also include a detergent or alcohol. The home care compositions that contain silver dihydrogen citrate can be formulated in a variety of ways, and may include both an aqueous and a non aqueous, or oil, phase, optionally including an emulsifying agent. Additionally the compositions may include gelling agents or thickening agents. Suitable physical forms include liquids, Semi-solids, pastes, gels, bars, tablets, sprays, foams, powders or granules.

The invention further provides methods of using home care compositions by applying them to an appropriate article or surface.

### DETAILED DESCRIPTION OF THE INVENTION

The invention described herein provides personal care and home care products having antimicrobial properties, including activity against bacteria, fungi and viruses. The consumer products of the invention contain an antimicrobially effective amount of silver dihydrogen citrate. Such antimicrobially effective amount is sufficient to either preserve the product of the invention against product degradation by microbes or to confer a beneficial effect on a person, article or environmental space or surface. Such a beneficial effect includes killing or preventing the proliferation of microbes on the article to which the product is applied.

As used herein, the term “silver dihydrogen citrate” refers to molecule having the chemical formula AgC₆H₇O₇. The chemical structure is principally: 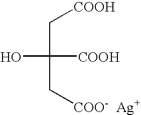
although the presence of lesser amounts of related compositions is not excluded.

Generally, silver dihydrogen citrate can be made by immersing silver electrodes in an aqueous electrolyte solution that contains citric acid. An electrolytic potential is then applied to the electrodes, whereby silver ion is generated in the solution. When combined in this way, silver ions and citric acid form silver dihydrogen citrate, which is stable in aqueous solution. In some embodiments of the invention, the electrolyte contains greater than about 5% and more particularly greater than about 10% citric acid (% wt/volume). The silver dihydrogen citrate can then be formulated or combined with other ingredients as further described herein. The invention provides personal care compositions that contain silver dihydrogen citrate and a physiologically acceptable medium. The silver dihydrogen citrate is prepared as described above.

Silver dihydrogen citrate has been shown to have antimicrobial activity against a variety of microbes, including bacteria, fungi and viruses. Particular microbes against which efficacy has been demonstrated include Pseudomonas aeruginosa (especially ATCC 15442), Salmonella choleraesuis (especially ATCC 10708), Staphylococcus aureus (especially ATCC 65328 and ATCC 700698), E. coli (especially 0157:H7, ATCC 43888 and ATCC 11229), Listeria monocytogene (especially ATCC 11543 and 19111), Enterococcus faecium (especially ATCC 6569 and ATCC 700221), human immunodeficiency virus 1 (HIV 1), herpes simplex virus type 1 (HSV 1), poliovirus type 2, influenza A, rhinovirus, Propionibacterium acnes (especially ATCC 6921), Trichophyton mentagrophytes (especially ATCC 9533). The personal care compositions and home care compositions of the invention possess preservative antimicrobial activity against these microbes and other microbes.

The invention provides personal care compositions containing silver dihydrogen citrate in a physiologically acceptable medium. As used herein “physiologically acceptable medium” means a composition which is non-toxic, non-irritating and otherwise suitable for contact with the surfaces of a human or other vertebrate body. Such surfaces include the hair, skin, mouth, anal, urethral and vaginal surfaces. Whether a composition is physiologically acceptable can be determined by tests well known to those of skill in the art.

Some appropriate personal care compositions of the invention include deodorants, antiperspirants, skin care products for facial, foot, hand and whole body uses, sun protection products, personal cleaning products, hair care products, feminine hygiene products, oral care products and decorative cosmetics such as lipsticks, mascara, facial makeup crèmes and rouge. Thus the products may include other appropriate agents such as moisturizers humectants, emollients, oils, lipid-type materials, stabilizers, abrasives, anti-acne agents, antioxidants, colorants, astringents, film formers, fragrance components, opacifying agents, propellants, reducing agents, skin bleaching agents, sunscreen agents, oral care agents, such as described herein. Alcohols and detergents may additionally be added.

The personal care compositions of the invention containing silver dihydrogen citrate can be provided in an aqueous phase As implied, the aqueous phase contains water and optionally contains other ingredients as described herein. Alternatively, the compositions can contain both an aqueous and an oil phase. In the latter case, they can include an emulsifying agent so as to create an emulsion, that is a dispersion of one liquid in another, in which the dispersed phase is stabilized by the emulsifying agent. An emulsion is generally a stable dispersion of a first liquid in a second immiscible liquid. The emulsifier is an amphoteric substance that prevents the dispersed liquid from coalescing. The invention provides water-in-oil emulsions, oil-in-water emulsions, oil-in-water-in-oil dispersions, water-in-oil-in-water emulsions, phase inversion temperature emulsions and microemulsions. Examples of emulsions are described in further detail herein.

The invention further provides personal care compositions containing silver dihydrogen citrate and a water-insoluble solid in an aqueous phase. In some embodiments, the water-insoluble solid is dispersed in the aqueous phase to form an aqueous colloidal suspension. In some embodiments, such aqueous colloidal suspensions further comprise an agent capable of stabilizing the solid as a suspension in the aqueous phase. In some embodiments, the water-insoluble solid is dispersed in an emulsion, or in a water-based gel. In some further compositions, the water-insoluble solid ingredient forms, along with the aqueous phase, a paste.

The term “water insoluble ingredient” means an ingredient whose concentration in the composition exceeds the solubility of such ingredient in water. As the term “water insoluble ingredient” is relative to the concentration of the ingredient in the composition, a water soluble ingredient used in a paste may be capable of dissolving in large excesses of water, but incapable of wholly dissolving in the amount of water used in the paste. On the other hand, a “water insoluble ingredient” used in an aqueous solution, or an aqueous phase of a colloidal composition, must be only very slightly soluble in water. The person skilled in the art will perceive these requirements and make appropriate selections based on the ordinary skill in the art.

The invention further provides personal care compositions that contain silver dihydrogen citrate, water and at least one member of the group consisting of gelling agents, thickeners and mixtures thereof.

The invention further provides methods of using the personal care compositions. In general, the method comprises applying the personal care composition to a body surface or part to be treated. The term “applying” includes an appropriate action on the part of the user to contact the personal care composition to the body part. Applying includes, in some embodiments, spreading, spraying, squirting, wiping and brushing. The particular type of application depends on the body part to which the personal care composition is to be applied.

“Body part” means a part of body including the mouth and other epithelial surfaces of the body. Thus the term body part includes hair, skin and mouth, anus, urethra and vagina. In the case of the skin, the body part is often more specific. For example, in some embodiments the body part is the skin of the face, hand or foot. In other embodiments, the body part is the whole body. In other embodiments, for example where the personal care compositions are deodorants or antiperspirants, body part can be the underarms.

The invention also provides home care compositions comprising silver dihydrogen citrate, water and at least one ingredient other than a detergent, alcohol or both.

The term “home care composition” means a composition for use in the general environment of human beings, and is further described herein. Home care compositions are generally non-toxic when applied in the vicinity of human being, for example to fabrics and other items used by humans, when applied to surfaces used by, or in the vicinity of, humans, or when applied to spaces occupied by humans.

The invention also provides home care compositions comprising silver dihydrogen citrate, water, an oil phase and an emulsifier. Such compositions are emulsions, that is dispersions of a first liquid phase in a second, immiscible liquid phase, wherein the emulsifier stabilizes the dispersion of the first phase in the second phase.

The invention also provides home care compositions containing a mixture of a water-insoluble ingredient in a water phase, wherein the water phase comprises water and silver dihydrogen citrate. Such mixtures include colloids and pastes as described further herein.

The invention also provides home care compositions containing silver dihydrogen citrate, water and at least one member of the group consisting of gelling agents and thickeners. Such compositions are gels, as described in more detail herein.

The invention also provides home care compositions containing silver dihydrogen citrate and water in liquid, semi-solid or solid form.

The invention also provides home care compositions comprising silver dihydrogen citrate and further comprising one or more additional ingredients selected from the group consisting of builders, enzymes, bleaches, whiteners, color care agents, fabric softeners, suds suppressors, dispersants, dye transfer inhibitors, chelating agents and aerosol propellants, each as described in further detail herein.

The invention also provides antimicrobial laundry care compositions in liquid, paste, gel, bar, tablet, spray, foam, powder or granule form, each as described in more detail herein.

The invention also provides home care methods comprising applying a home care composition to an article, surface or space. Exemplary articles, surfaces and spaces include clothes, furniture fabrics, rugs and carpets, draperies, dishes and cooking utensils, grills, ovens, and other items used by humans.

The term “surface” includes hard surfaces in the human environment, such as floors, glass surfaces (such as glass windows, doors and countertops), other counter surfaces, bath, toilet bowl, sink and other bathroom surfaces.

The term “space” includes the interior portion of buildings occupied by humans, including the air contained therein.

The invention provides personal care compositions containing silver dihydrogen citrate present at a concentration effective to preserve the composition against microbes. The invention further provides personal care compositions containing sufficient silver dihydrogen citrate to confer an antimicrobial effect on a person to whom it is applied. Such personal care compositions are non-toxic, cost-effective and shelf-stable over prolonged periods. Such personal care compositions can include: underarm deodorant sprays, roll-ons, sticks and gels; underarm antiperspirant sprays, roll-ons, sticks and gels; underarm antiperspirant/deodorant sprays, roll-ons, sticks and gels; skin treatment creams, lotions, gels, tonics, sprays and oils; sun-protectant creams, lotions, gels, sprays and oils; personal cleansing (hand and/or body) soaps, shampoos, bath oils and beads; hair shampoos, rinses, conditioners, gels, oils and/or dyes; oral care products (dentrifices) such as toothpastes, tooth gels, oral rinses, whitening compositions and dental flosses; and cosmetic lotions, sticks, creams, oils and ointments.

The invention further provides home or industrial care compositions for treating articles, surfaces or spaces in the human environment. In some embodiments, such home or industrial care compositions possess effective antimicrobial preservative properties. In other embodiments, such home or industrial care compositions confer an antimicrobial effect on articles, surfaces or spaces to which they are applied. Home and industrial care compositions provided by the invention include: surface cleaning compositions (for example, glass, floor, counter, bath, toilet bowl, sink, appliance and furniture cleaning compositions); deodorants (for example, solid, liquid and spray deodorants air and/or surface deodorants); disinfectants (for example, spray and solid (including gel) air disinfectants; and spray, solid, liquid and paste surface disinfectants); waxes and other surface protecting and/or polishing compositions; laundry compositions (for example detergents, fabric softeners and whiteners); and rug shampoos.

In general, preparation of aqueous silver dihydrogen citrate can be accomplished as follows. First, an aqueous electrolyte solution comprising an appropriate concentration of citric acid is prepared by mixing citric acid with an appropriate amount of water to form an electrolyte solution. The electrolyte solution is also referred to herein as the “aqueous citric acid” or “aqueous citric acid solution.” The electrolyte solution is then exposed to a pair of silver electrodes. In some embodiments the anode is at least about 99.9% pure Ag⁰. In some embodiments, both anode and cathode are at least about 99.9% pure Ag⁰. In some embodiments, pure Ag⁰ refers to about 99.99% pure Ag⁰, 99.999% pure Ag⁰ or 99.9999% pure Ag⁰. In some embodiments, the anode may be made of a higher purity elemental silver (Ag⁰) than the cathode. A potential difference of about 12 to 50 volts is applied between the anode and cathode, whereby a current flows between the two electrodes, and silver ion (Ag⁺) is released into the aqueous citric acid.

The silver ion (Ag⁺) is stabilized in the aqueous citric acid. In some embodiments, the concentration of citric acid required to obtain a concentration of aqueous silver ion of about 0.1% silver ion is about 10% acid by volume. An advantageous range of concentrations for the citric acid in the electrolyte is about 10% to about the solubility limit of citric acid in water (i.e. about 52 g citric acid per 100 g of water at 20° C.).

It has been found that, in general, the greater the concentration of citric acid in solution, the greater the concentration of silver ion that is obtainable in the solution. For example, while it has been shown that it is possible to obtain a silver ion concentration of about 0.1% Ag⁺ in a 10% aqueous citric acid solution, lower concentrations of Ag⁺ ion can be obtained using lower concentrations of citric acid, whereas higher concentrations are obtainable using higher concentrations of citric acid. It is thus possible to adjust the upper limit of the silver ion concentration in the aqueous citric acid by varying the amount of citric acid in the electrolyte solution, for example up to the maximum solubility of citric acid in water. It is likewise possible to adjust the final silver ion concentration in the aqueous citric acid, up to such upper limit, by varying the potential difference and/or the current flow between the electrodes, as well as the length of time that the voltage is applied to the electrodes while they are exposed to the electrolyte.

In some embodiments, the aqueous silver dihydrogen citrate is combined with one or more additional ingredients such as described in more detail herein. The invention provides silver dihydrogen citrate compositions in the form of liquids, colloids, liposomes, gels, sols, pastes, lotions, ointments, oils, and other physical forms. An advantage of the inventive silver dihydrogen citrate solution is that it provides stabilized silver ion in an antimicrobially effective concentration over a relatively long period of time, for example days, months or years. Thus, the inventive compositions comprising such silver dihydrogen citrate possess antimicrobial preservative characteristics. Additionally, in some embodiments, the silver dihydrogen citrate is present in the inventive composition at a concentration effective to confer an antimicrobial effect on the user's person or environment (for example air, a surface or clothing). Thus, in some embodiments, the inventive compositions possess the advantage of conferring on the user or the user's environment an enhanced, improved or qualitatively different antimicrobial effect than has been heretofore available.

The invention provides antimicrobially effective silver dihydrogen citrate compositions comprising silver ion at a concentration of at least about 50 parts per billion (ppb), especially at least about 100 ppb, and more specifically at least about 1 part per million (ppm). Exemplary antimicrobially effective ranges of silver ion concentration are from about 50 ppb to about 10,000 ppm, especially about 100 ppb to about 2,400 ppm, and more specifically about 1 ppm to about 1,000 ppm. These concentrations are based on the weight of silver ion per unit volume of the final composition (if liquid) or per unit weight of the final composition (if solid).

The term “silver dihydrogen citrate” as used herein is distinguished from other antimicrobial agents which may be added to the inventive compositions in some embodiments.

The silver dihydrogen citrates of the present invention are used alone or, in some embodiments, in combination with one or more other antimicrobials and/or biocides, for example as in-can preservatives for microbe-susceptible water-containing products (such as raw materials for cosmetic and pharmaceutical products and for preservation of personal care products, cosmetic products, toiletries, oral care products, pharmaceutical products, household products such as surface cleaners, softeners and detergents, industrial and institutional products) and other products containing water that need to be preserved to avoid microbial spoilage.

In some inventive embodiments, cosmetic formulations or pharmaceutical compositions that contain one or more silver dihydrogen citrates according to the present invention may additionally contain one or more further antimicrobial agents as listed below.

In some embodiments, antimicrobial preparations are prepared by physically mixing the inventive silver dihydrogen citrate (and optionally other antimicrobial agents) with another active or inert substance using customary methods, for example by simply stirring together the individual components, especially by making use of the dissolution properties of already known antimicrobial agents.

In some embodiments, inventive cosmetic or pharmaceutical preparations contain from 0.05-95% (volume/volume) of the silver dihydrogen citrate-containing silver dihydrogen citrate according to the present invention. Some inventive cosmetic compositions also contain other antimicrobial agents or mixtures of antimicrobial agents in addition to the silver dihydrogen citrate silver dihydrogen citrate.

An “antimicrobial agent” is an agent capable of eliciting an antimicrobial effect. The invention provides antimicrobial agents that have bacteriostatic, bacteriocidal, virucidal, virustatic, fungicidal or fungistatic activities. An “antimicrobial agent other than an alcohol” is an antimicrobial agent other than one of the mono-hydroxy compounds conventionally known as alcohols, such as ethanol, isopropanol, isobutanol and phenol.

Examples of additional antimicrobial agents that are employed in some embodiments of the present invention include: Pyrithiones, especially the zinc complex (ZPT); Octopirox®; Dimethyldimethylol Hydantoin (Glydant®); Methylchloroisothiazolinone/methylisothiazolinone (Kathon CG®); Sodium Sulfite; Sodium Bisulfite; Imidazolidinyl Urea (Germall 115®, Diazolidinyl Urea (Germaill II®); Benzyl Alcohol; 2-Bromo-2-nitropropane-1,3-diol (Bronopol®); Formalin (formaldehyde); lodopropenyl Butylcarbamate (Polyphase P100®); Chloroacetamide; Methanamine; Methyldibromonitrile Glutaronitrile (1,2-Dibromo-2,4-dicyanobutane or Tektamer®); Glutaraldehyde; 5-bromo-5-nitro-1,3-dioxane (Bronidox®); Phenethyl Alcohol; o-Phenylphenol/sodium o-phenylphenol; Sodium Hydroxymethylglycinate (Suttocide A®); Polymethoxy Bicyclic Oxazolidine (Nuosept C®); Dimethoxane; Thimersal; Dichlorobenzyl Alcohol; Captan; Chlorphenenesin; Dichlorophene; Chlorbutanol; Glyceryl Laurate; Halogenated Diphenyl Ethers; 2,4,4′-trichloro-2′-hydroxy-diphenyl ether (Triclosan®. or TCS); 2,2′-dihydroxy-5,5′-dibromo-diphenyl ether; Phenolic Compounds; Phenol; 2-Methyl Phenol; 3-Methyl Phenol; 4-Methyl Phenol; 4-Ethyl Phenol; 2,4-Dimethyl Phenol; 2,5-Dimethyl Phenol; 3,4-Dimethyl Phenol; 2,6-Dimethyl Phenol; 4-n-Propyl Phenol; 4-n-Butyl Phenol; 4-n-Amyl Phenol; 4-tert-Amyl Phenol; 4-n-Hexyl Phenol; 4-n-Heptyl Phenol; Mono- and Poly-Alkyl and Aromatic Halophenols; p-Chlorophenol; Methyl p-Chlorophenol; Ethyl p-Chlorophenol; n-Propyl p-Chlorophenol; n-Butyl p-Chlorophenol; n-Amyl p-Chlorophenol; sec-Amyl p-Chlorophenol; Cyclohexyl p-Chlorophenol; n-Heptyl p-Chlorophenol; n-Octyl p-Chlorophenol; o-Chlorophenol; Methyl o-Chlorophenol; Ethyl o-Chlorophenol; n-Propyl o-Chlorophenol; n-Butyl o-Chlorophenol; n-Amyl o-Chlorophenol; tert-Amyl o-Chlorophenol; n-Hexyl o-Chlorophenol; n-Heptyl o-Chlorophenol; o-Benzyl p-Chlorophenol; o-Benxyl-m-methyl p-Chlorophenol; o-Benzyl-m; m-dimethyl p-Chlorophenol; o-Phenylethyl p-Chlorophenol; o-Phenylethyl-m-methyl p-Chlorophenol; 3-Methyl p-Chlorophenol; 3,5-Dimethyl p-Chlorophenol; 6-Ethyl-3-methyl p-Chlorophenol; 6-n-Propyl-3-methyl p-Chlorophenol; 6-iso-Propyl-3-methyl p-Chlorophenol; 2-Ethyl-3,5-dimethyl p-Chlorophenol; 6-sec-Butyl-3-methyl p-Chlorophenol; 2-iso-Propyl-3,5-dimethyl p-Chlorophenol; 6-Diethylmethyl-3-methyl p-Chlorophenol; 6-iso-Propyl-2-ethyl-3-methyl p-Chlorophenol; 2-sec-Amyl-3,5-dimethyl p-Chlorophenol; 2-Diethylmethyl-3,5-dimethyl p-Chlorophenol; 6-sec-Octyl-3-methyl p-Chlorophenol; p-Chloro-m-cresol: p-Bromophenol; Methyl p-Bromophenol; Ethyl p-Bromophenol; n-Propyl p-Bromophenol; n-Butyl p-Bromophenol; n-Amyl p-Bromo-phenol; sec-Amyl p-Bromophenol; n-Hexyl p-Bromophenol; Cyclohexyl p-Bromophenol; o-Bromophenol; tert-Amyl o-Bromophenol; n-Hexyl o-Bromophenol; n-Propyl-m,m-Dimethyl o-Bromophenol; 2-Phenyl Phenol; 4-Chloro-2-methyl phenol; 4-Chloro-3-methyl phenol; 4-Chloro-3,5-dimethyl phenol; 2,4-Dichloro-3,5-dimethylphenol; 3,4,5,6-Terabromo-2-methyl-phenol; 5-Methyl-2-pentylphenol; 4-Isopropyl-3-methylphenol; Para-chloro-meta-xylenol (PCMX); Chlorothymol; Phenoxyethanol; Phenoxyisopropanol; 5-Chloro-2-hydroxydiphenyl-methane; Resorcinol and its Derivatives; Resorcinol; Methyl Resorcinol; Ethyl Resorcinol; n-Propyl Resorcinol; n-Butyl Resorcinol; n-Amyl Resorcinol; n-Hexyl Resorcinol; n-Heptyl Re-sorcinol; n-Octyl Resorcinol; n-Nonyl Resorcinol; Phenyl Resorcinol; Benzyl Resorcinol; Phe-nylethyl Resorcinol; Phenylpropyl Resorcinol; p-Chlorobenzyl Resorcinol; 5-Chloro 2,4-Dihy-droxydiphenyl Methane; 4′-Chloro 2,4-Dihydroxydiphenyl Methane; 5-Bromo 2,4-Dihydroxy-diphenyl Methane; 4′-Bromo 2,4-Dihydroxydiphenyl Methane; Bisphenolic Compounds; 2,2′-Methylene bis-(4-chlorophenol); 2,2′-Methylene bis-(3,4,6-trichlorophenol); 2,2′-Methylene bis-(4-chloro-6-bromophenol); bis(2-hydroxy-3,5-dichlorophenyl)sulfide; bis(2-hydroxy-5-chloro-benzyl)sulfide; Benzoic Esters (Parabens); Methylparaben; Propylparaben; Butylparaben; Ethylparaben; Isopropylparaben; Isobutylparaben; Benzylparaben; Sodium Methylparaben; Sodium Propylparaben; Halogenated Carbanilides; 3,4,4′-Trichlorocarbanilides (Triclocarban® or TCC); 3-Trifluoromethyl-4,4′-dichlorocarbanilide; 3,3′,4-Trichlorocarbanilide; Chlorohexidine and its digluconate; diacetate and dihydrochloride; Undecenoic acid;. Hexetidine; poly(hexamethylenebiguanide) hydrochloride (Cosmocil®).

For the purpose of preservation of cosmetic, pharmaceutical, household and technical products, combinations of the silver dihydrogen citrate-containing silver dihydrogen citrate of the present invention with other antimicrobial preservatives such as those of the Annex VI of the European Cosmetic Directive (listed in the following) show increased preservative efficacy: formaldehyde; paraformaldehyde; hydroxy biphenyl and its salts such as ortho-phenylphenol; zinc pyrithion; chlorobutanol; hydroxy benzoic acids and their salts and esters such as methyl paraben, ethyl paraben, propyl paraben, butyl paraben; dibromo hexamidine and its salts including isethionate (4,4′-hexamethylenedioxy-bis(3-bromo-benzamidine) and 4,4′-hexamethylenedioxy-bis(3-bromo-benzamidinium 2-hydroxyethanesulfonate); mercury, (aceto-O)phenyl (i.e. phenyl mercuric acetate) and Mercurate(2-),(orthoboate(3-)-O)phenyl, dihydrogene (i.e. phenyl mercuric borate); 1,3-bis(2-ethylhexyl)-hexahydro-5-methyl-5-pyrimidine (Hexetidin); 5-bromo-5-nitro-1,3-dioxan; 2-bromo-2-nitro-1,3-propandiol; 2,4-dichlorobenzyl alcohol; 3,4,4′trichlorocarbanilide (Trichlorcarban); p-chloro-m-cresol; 2,4,4′-trichloro 2-hydroxy diphenylether (triclosan); 4,4′-dichloro 2-hydroxy diphenylether; 4-chloro-3,5-dimethylphenol (Chloroxylenol); imidazolidinyl urea; poly-(hexamethylene biguanide) hydrochloride; 2-phenoxy ethanol (phenoxyethanol); hexamethylene tetramine (Methenamine); 1-(3-chloroallyl)-3,5,7-triaza-1-azonia-adamantanchloride (Quatemium 15); 1-(4-chlorophenyoxy)-1-(1-imidazolyl)3,3-dimethyl-2-butanone (Climbazole); 1,3-bis(hydroxymethyl)-5,5-dimethyl-2,4-imidazolidinedione (DMDM hydantoin); benzyl alcohol; 1,2-dibromo-2,4-dicyano butane; 2,2′methylene-bis(6-bromo-4-chloro phenol) (bromochlorophene); methylchloroisothiazolone, methylisothiazolone, octylisothiazolone, benzylisothiazolone; 2-benzyl-4-chlorophenol (Chlorophenone); chloracetamide; chlorhexidine, chlorhexidine acetate, chlorhexidine gluconate, chlorhexidine hydrochloride; 1-phenoxy-propane-2-ol (phenoxyisopropanol); 4,4-dimethyl-1,3-oxazolidine (dimethyl oxazolidine); diazolidinyl urea; 4,4′-hexamethylenedioxybisbenzamidine and 4,4′-hexamethylenedioxybis(benzamidinium-2-hydroxyethanesulfonate); glutaraldehyde (1,5-pentanedial); 7-ethylbicyclooxazolidine; 3-(4-chlorophenoxy)-1,2-propandiol (chlorophenesin); phenylmethoxymethanol and ((phenylmethoxy)methoxy)-methanol (benzylhemiformal); N-alkyl(C12-C22)trimethyl ammoniumbromide and -chloride (cetrimonium bromide, cetrimonium chloride); benzyl-dimethyl-(4-(2-(4-(1,1,3,3-tetramethylbutyl)-phenoxy)-ethoxy)-ethyl)-ammonium chloride (benzethonium chloride); Alkyl-(C8-C18)-dimethyl-benzylammonium chloride, -bromide and saccharinate; (benzalkonium chloride, benzalkonium bromide, benzalkonium saccharinate); benzoic acid and its salts and esters; propionic acid and its salts; salicylic acid and its salts; sorbic acid and its salts; sodium iodinate; anorganic sulfites and bisulfites such as sodium sulfite; dehydroacetic acid; formic acid; mercurate(1-ethyl)2-mercaptobenzoate(2-)-O,S-,hydrogene (Thiomersal or Thiomerosal); 10-undecylenic acid and its salts; octopirox (piroctone olamine); sodium hydroxy methyl-aminoacetate (sodium hydroxymethylglycinate); and 3-iodo-2-propynyl butylcarbamate.

The invention further provides compositions contain other antibacterial agents, which include natural antibacterial actives that are natural essential oils or derivatives thereof. These agents derive their names from their natural occurrence in plants, microorganisms or animals. Some natural essential oils having antibacterial properties include oils of anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, blue cypress, gum benzoin, basil, fennel, fir, balsam, menthol, ocmea origanum, Hydastis carradensis, Berberidaceae daceae, Ratanhiae and Curcuma longa. Also included in this class of natural essential oils are the key chemical components of the plant, microbial or animal-derived oils which have been found to provide the antimicrobial benefit. These chemicals include, but are not limited to anethol, catechole, camphene, carvacol, eugenol, eucalyptol, ferulic acid, farnesol, hinokitiol, tropolone, limonene, menthol, methyl salicylate, thymol, terpineol, verbenone, berberine, ratanhiae extract, caryophellene oxide, citronellic acid, curcumin, nerolidol and geraniol, chitosans or their derivatives.

The invention further provides compositions containing non-silver antibacterial metal salts. This class generally includes salts of metals in groups 3b-7b, 8 and 3a-5a. Specifically useful in some embodiments of the invention are the salts of aluminum, zirconium, zinc, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof.

The invention further provides compositions that comprise one or more chelating agents. Exemplary chelating agents are ethylene di-amine tetra acetic acid (EDTA), beta-alanine diacetic acid (EDETA), phosphonomethyl chitosan, carboxymethyl chitosan, hydroxyethylene di-amino tetraacetic acid, nitrilotriacetic acid (NTA) and ethylenediamine disuccinic acid (S,S-EDDS, R,R-EDDS or S,R-EDDS). In some embodiments, such chelating agents provide additional or even synergistic effects in combination with the inventive silver dihydrogen citrate silver dihydrogen citrate.

The invention further provides personal care and home care compositions that comprise one or more fragrances. In particular compositions, the combination of the silver dihydrogen citrate silver dihydrogen citrate with one or more perfumes, particularly those containing plant derived oils, result in improved or qualitatively different antimicrobial efficacy.

The invention also provides hair care compositions and methods that confer an anti-dandruff effect, particularly by combining silver dihydrogen citrate silver dihydrogen citrates with other antimicrobials such as zinc pyrithion, octopirox, climbazol, sulfur, imidazole derivatives such as ketoconazole and itraconazole or salicylic acid.

The invention provides deodorant personal care and home care compositions, such as underarm deodorants, underarm antiperspirant/deodorant compositions, aerosol room deodorizers, solid room deodorizers, etc. In particular, the invention provides personal care and home care deodorant compositions comprising one or more members of following group: farnesol, perfumes, phenoxyethanol, quaternary compounds, triclosan, triclocarban, organic acids such as benzoic acid or sorbic acid, biguanides such as poly-(hexamethylene biguanide) hydrochloride or any other silver dihydrogen citrate listed above. The invention further provides personal care compositions comprising a deodorant and one or more antiperspirant agents (for example aluminum chlorhydrate, zirconium chlorhydrate and other salts of aluminum, zinc and zirconium), alcohol, chelating agents or antioxidants. In some embodiments, such ingredients also result in enhanced or qualitatively different antimicrobial activity for the inventive silver dihydrogen citrate.

The invention further provides personal care compositions and methods effective against dermatophytes. Dermatophytes are parasitic fungi that infect skin, hair or nails. For example combination of a silver dihydrogen citrate silver dihydrogen citrate of the present invention with 10-undecylenic acid, sorbic acid, benzoic acid, salicylic acid, or imidazole derivatives such as ketoconazole and/or itraconazole, will, in some embodiments, give rise to an enhanced dermatophytically effective composition. Applying such a composition to a locus of dermatophyte infestation will result in arrest of dermatophyte proliferation, decrease in severity of the infection, or both.

The invention further provides personal care compositions and methods effective against acne. For example combination of the inventive silver dihydrogen citrate with other antimicrobials such as phenoxyethanol, phenoxypropanol, benzalkonium chloride, cetrimonium bromide or benzethonium chloride, sulfur or salicylic acid, will give rise to an anti-acne composition. When applied to an area affected by acne, the composition will give rise to an anti-acne effect.

The invention further provides a cleansing composition that comprises an anionic surfactant. In some embodiments, the anionic surfactant constituted from about 0.05% to about 10%, preferably from about 0.1% to about 2%, and more preferably from about 0.2% to about 1%, by weight of the cleansing composition.

Non-limiting examples of anionic lathering surfactants useful in embodiments of the compositions of the present invention are disclosed in McCutcheon's, Detergents and Emulsifiers, North American edition (1990), published by The Manufacturing Confectioner Publishing Co.; McCutcheon's, Functional Materials, North American Edition (1992); and U.S. Pat. No. 3,929,678, to Laughlin et al., issued Dec. 30, 1975, all of which are incorporated herein by reference.

A wide variety of anionic surfactants will be useful in embodiments of the invention. Non-limiting examples of anionic lathering surfactants include those selected from the group consisting of alkyl and alkyl ether sulfates; sulfated monoglycerides; sulfonated olefins; alkyl aryl sulfonates; primary or secondary alkane sulfonates; alkyl sulfosuccinates; acyl taurates; acyl isethionates; alkyl glycerylether sulfonate; sulfonated methyl esters; sulfonated fatty acids; alkyl phosphates; acyl glutamates; acyl sarcosinates; alkyl sulfoacetates; acylated peptides; alkyl ether carboxylates; acyl lactylates; anionic fluorosurfactants; and mixtures thereof. Mixtures of anionic surfactants can be used effectively in some embodiments of the present invention.

Anionic surfactants for use in inventive cleansing compositions include alkyl and alkyl ether sulfates. These materials have the respective formulae R₁₁—O—SO₃-M and R₁₁—(CH₂H₄—O)ₓ—O—SO₃-M, wherein R₁₁ is a saturated or unsaturated, branched or unbranched alkyl group from about 8 to about 24 carbon atoms, x is 1 to 10, and M is a water-soluble cation such as ammonium, sodium, potassium, magnesium, triethanolamine, diethanolamine and monoethanolamine. The alkyl sulfates are typically made by the sulfation of monohydric alcohols (having from about 8 to about 24 carbon atoms) using sulfur trioxide or other known sulfation technique. The alkyl ether sulfates are typically made as condensation products of ethylene oxide and monohydric alcohols (having from about 8 to about 24 carbon atoms) and then sulfated. These alcohols can be derived from fats, for example, coconut oil or tallow, or can be synthetic. Specific examples of alkyl sulfates which are useful in some embodiments of inventive cleanser compositions are sodium, ammonium, potassium, magnesium, or TEA salts of lauryl or myristyl sulfate. Examples of alkyl ether sulfates include ammonium, sodium, magnesium, or TEA laureth-3 sulfate.

Another suitable class of anionic surfactants are the sulfated monoglycerides of the formula R₁₂—CO—O—CH₂—C(OH)H—CH₂—O—SO₃-M, wherein R₁₂ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are typically made by the reaction of glycerin with fatty acids (having from about 8 to about 24 carbon atoms) to form a monoglyceride and the subsequent sulfation of this monoglyceride with sulfur trioxide. An example of a sulfated monoglyceride is sodium cocomonoglyceride sulfate.

Other suitable anionic surfactants include olefin sulfonates of the form R₁₃SO₃-M, wherein R₁₃ is a mono-olefin having from about 12 to about 24 carbon atoms, and M is a water-soluble cation such as ammonium, sodium, potassium, magnesium, triethanolamine, diethanolamine and monoethanolamine. These compounds can be produced by the sulfonation of α-olefins by means of uncomplexed sulfur trioxide, followed by neutralization of the acid reaction mixture in conditions such that any sulfones which have been formed in the reaction are hydrolyzed to give the corresponding hydroxyalkanesulfonate. An example of a sulfonated olefin is sodium C₁₄/C₁₆ α-olefin sulfonate.

Other suitable anionic surfactants are the linear alkylbenzene sulfonates of the form R₁₄—C₆H₄—SO₃-M, wherein R₁₄ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are formed by the sulfonation of linear alkyl benzene with sulfur trioxide. An example of this anionic surfactant is sodium dodecylbenzene sulfonate.

Still other anionic surfactants suitable for embodiments of inventive the cleansing composition include the primary or secondary alkane sulfonates of the form R₁₅—SO₃-M, wherein R₁₅ is a saturated or unsaturated, branched or unbranched alkyl chain from about 8 to about 24 carbon atoms, and M is a water-soluble cation such as ammonium, sodium, potassium, magnesium, triethanolamine, diethanolamine and monoethanolamine. These are commonly formed by the sulfonation of paraffins using sulfur dioxide in the presence of chlorine and ultraviolet light or another known sulfonation method. The sulfonation can occur in either the secondary or primary positions of the alkyl chain. An example of an alkane sulfonate useful herein is alkali metal or am monium C₁₃-C₁₇ paraffin sulfonates.

Still other suitable anionic surfactants are the alkyl sulfosuccinates, which include disodium N-octadecylsulfosuccinamate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

Also useful are taurates which are based on taurine, which is also known as 2-aminoethanesulfonic acid. Examples of taurates include N-alkyltaurines such as the one prepared by reacting dodecylamine with sodium isethionate according to the teaching of U.S. Pat. No. 2,658,072 which is incorporated herein by reference in its entirety. Other examples of taurine derivatives that are useful in embodiments of the invention include the acyl taurines formed by the reaction of n-methyl taurine with fatty acids (having from about 8 to about 24 carbon atoms).

Another class of anionic surfactants suitable for use in some embodiments of the inventive cleansing composition are the acyl isethionates. The acyl isethionates typically have the formula R₁₆—CO—O—CH₂—CH₂SO₃-M, wherein R₁₆ is a saturated or unsaturated, branched or unbranched alkyl group having from about 10 to about 30 carbon atoms, and M is a cation. These are typically formed by the reaction of fatty acids (having from about 8 to about 30 carbon atoms) with an alkali metal isethionate. Nonlimiting examples of these acyl isethionates include ammonium cocoyl isethionate, sodium cocoyl isethionate, sodium lauroyl isethionate, and mixtures thereof.

Still other suitable anionic surfactants are the alkylglyceryl ether sulfonates of the form R₁₇—OCH₂—C(OH)H—CH₂—SO₃-M, wherein R₁₇ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These can be formed by the reaction of epichlorohydrin and sodium bisulfite with fatty alcohols (having from about 8 to about 24 carbon atoms) or other known methods. One example is sodium cocoglyceryl ether sulfonate.

Other suitable anionic surfactants include the sulfonated fatty acids of the form R₁₈—CH(SO₄)—COOH and sulfonated methyl esters of the from R₁₈—CH(SO₄)—CO—O—CH₃, where R₁₈ is a saturated or unsaturated, branched or unbranched alkyl group from about 8 to about 24 carbon atoms. These surfactants are generally formed by the sulfonation of fatty acids or alkyl methyl esters (having from about 8 to about 24 carbon atoms) with sulfur trioxide or by other known sulfonation techniques. Examples include alpha sulfonated coconut fatty acid and lauryl methyl ester.

Other suitable anionic materials include phosphates such as monoalkyl-, dialkyl-, and trialkylphosphate salts formed by the reaction of phosphorous pentoxide with monohydric branched or unbranched alcohols having from about 8 to about 24 carbon atoms. In some embodiments, these anionic materials are also be formed by other known phosphation methods. An example from this class of surfactants is sodium mono or dilaurylphosphate.

Other suitable anionic materials include acyl glutamates corresponding to the formula R₁₉—CO—N(COOH)—CH₂CH₂—CO₂-M wherein R₁₉ is a saturated or unsaturated, branched or unbranched alkyl or alkenyl group of about 8 to about 24 carbon atoms, and M is a water-soluble cation. Nonlimiting examples of which include sodium lauroyl glutamate and sodium cocoyl glutamate.

Other anionic materials include alkanoyl sarcosinates corresponding to the formula R₂₀—CON(CH₃)—CH₂CH₂—CO₂-M wherein R₂₀ is a saturated or unsaturated, branched or unbranched alkyl or alkenyl group of about 10 to about 20 carbon atoms, and M is a water-soluble cation. Nonlimiting examples of which include sodium lauroyl sarcosinate, sodium cocoyl sarcosinate, and ammonium lauroyl sarcosinate.

Other anionic materials include alkyl ether carboxylates corresponding to the formula R₂₁—(OCH₂CH₂)_{X}—OCH₂—CO₂-M wherein R₂₁ is a saturated or unsaturated, branched or unbranched alkyl or alkenyl group of about 8 to about 24 carbon atoms, x is 1 to 10, and M is a water-soluble cation. Nonlimiting examples of which include sodium laureth carboxylate.

Other anionic materials include acyl lactylates corresponding to the formula R₂₂—CO—[O—CH(CH₃)—CO]_{X}—CO₂-M wherein R₂₂ is a saturated or unsaturated, branched or unbranched alkyl or alkenyl group of about 8 to about 24 carbon atoms, x is 3, and M is a water-soluble cation, nonlimiting examples of which include sodium cocoyl lactylate.

Other anionic materials include the carboxylates, nonlimiting examples of which include sodium lauroyl carboxylate, sodium cocoyl carboxylate, and ammonium lauroyl carboxylate. Anionic flourosurfactants can also be used.

A counter cation, M, counterbalances the negative charge of the anionic surfactant. Some especially suitable counter cations are sodium, potassium, ammonium, monoethanolamine, diethanolamine, and triethanolamine. An especially suitable counter cation is ammonium.

The invention further provides personal care and home care compositions that comprise one or more non-ionic surfactants. Some nonionic surfactants are condensation products of ethylene oxide with various reactive hydrogen-containing compounds reactive therewith having long hydrophobic chains (for example aliphatic chains of about 12-20 carbon atoms), which condensation products (“ethoxamers”) contain hydrophilic polyoxyethylene moieties, such as condensation products of poly(ethyleneoxide) with fatty acids, fatty alcohols, fatty amides, polyhydric alcohols (for example sorbitan monostearate) and polypropylene oxide (for example Pluronic® materials). Polyoxamers include for example block copolymers of polyoxyethylene and polyoxypropylene having an average molecular weight from about 3000 to 5000 and a preferred average molecular weight from about 3500 to 4000 and containing about 10-80% hydrophilic polyoxyethylene groups, by weight, of the block copolymer (for example Pluronic F127).

The invention further provides personal care and home care compositions comprising one or more amphoteric surfactants. Some amphoteric surfactants are C₈-C₁₈-betains, C₈-C₁₈-sulfobetains, C₈-C₂₄-alkylamido-C₁-C₄-alkylene betains, imidazoline carboxylates, alkylampho-carboxycarbonic acids, alkylamphocarbonic acid (for example lauroamphoglycinate) and N-alkyl-β-aminopropionate or -iminodipropionate. In particular embodiments, the amphoteric surfactant comprises C₁₀-C₂₀-alkylamidoC₁-C₄-alkylenbetaine and/or coco fatty acid amide propylbetaine.

The invention further provides personal care and home care compositions comprising a combination of anionic, non-ionic and amphoteric surfactants. The anionic, non-ionic and amphoteric surfactants are set forth above.

The invention further provides antimicrobial compositions that comprise an additional proton donating agent (aside from citric acid, which itself could be considered a proton-donating agent), preferably from about 0.1% to about 10%, more preferably from about 0.5% to about 8%, and most preferably from about 1% to about 5% (based on the weight of the composition) of a proton donating agent. In this context “proton donating agent” means any acid compound or mixture thereof (aside from citric acid), which results in undissociated acid on the skin after use. Proton donating agents can be organic acids, including polymeric acids, mineral acids or mixtures thereof. These additional organic proton donating agents can be added directly to the composition in the acid form or can be formed by adding the conjugate base of the desired acid and a sufficient amount of a separate acid (for example the aforementioned organic acid) that is strong enough (i.e. has a low enough pKa) to form the undissociated acid from its conjugate base. In some embodiments, the proton donating agent includes a mineral acid that will not remain undissociated in the neat composition and/or when the composition is diluted during washing and rinsing. These proton donating agents are added directly to the composition in the acid form.

The invention further provides personal care and home care compositions having a pH in the range of about 2 to about 7. The silver dihydrogen citrate of the present invention is active within a broad pH range typically used in personal care and household products. The pH range of the formulation containing the silver dihydrogen citrate of the present invention is generally below pH 8. In some embodiments of the invention (including skin treatments and cleansers), the pH of the antimicrobial compositions of the present invention is adjusted to a sufficiently low level in order to either form or deposit substantial undissociated acid on the skin. In such embodiments, the pH of the present composition will be adjusted and preferably buffered to a range from about 3.0 to about 6.0, preferably from about 3.0 to about 5.0 and more preferably from about 3.5 to about 4.5. If necessary, strong acid (for example a mineral acid, such as HCl, H₂SO₄, H₃PO₄, HBr, HI, H₂SO₃, H₃PO₃, etc.) may be used to lower the pH into the desired range.

A non-exclusive list of examples of organic acids which act as the proton donating agent in some embodiments of the invention are adipic acid; tartaric acid; citric acid; maleic acid; lactic acid; malic acid; succinic acid; glycolic acid; glutaric acid; benzoic acid; malonic acid; salicylic acid; gluconic acid; gluconolactone (especially glucono-delta-lactone); 2-pyrrolidone-5 carboxylic acid; polyacrylic acid; salts thereof; and mixtures thereof (optionally with one or more mineral acids). A non-exclusive list of examples of mineral acid includes: hydrochloric; phosphoric; sulfuric and mixtures thereof. Particular examples of additional proton donating agents include: 2-pyrrolidone-5 carboxylic acid; gluconolactone; isomers thereof; and mixtures thereof.

The invention further provides personal care and home care compositions having “mildness-enhancing agents” added thereto. These “mildness-enhancing ingredients” include cationic and nonionic polymers, co-surfactants, moisturizers and mixtures thereof. Polymers used in some embodiments include: polyethylene glycols; polypropylene glycols; hydrolyzed silk proteins; hydrolyzed milk proteins; hydrolyzed keratin proteins; guar hydroxypropyltrimonium chloride; polyquats; silicone polymers and mixtures thereof. In some embodiments, the mildness enhancing polymers comprise from about 0.1% to about 1%, preferably from about 0.2% to about 1.0%, and more preferably from about 0.2% to about 0.6%, by weight of the antimicrobial composition. Co-surfactants used in some embodiments include: nonionic surfactants such as the Genapo124® series of ethoxylated alcohols; POE(20) sorbitan monooleate (Tween® 80); polyethylene glycol cocoate and Pluronic® propylene oxide/ethylene oxide block polymers; and amphoteric surfactants such as alkyl betaines; alkyl sultaines; alkyl amphoacetates; alkyl amphodiacetates; alkyl amphopropionates; and alkyl amphodipropionates. In some embodiments, the mildness enhancing co-surfactants comprise from about 20% to about 70%, preferably from about 20% to about 50%, by weight of the anionic surfactant, of the cleansing composition.

The invention further provides compositions comprising one or more lipid skin moisturizing agents, which provide a moisturizing benefit to the user when deposited to the user's skin. In some embodiments, lipophilic skin moisturizing agents are constitute about 0.1% to about 30%, preferably from about 0.2% to about 10%, most preferably from about 0.5% to about 5% by weight of the composition. In some embodiments, the lipophilic skin moisturizing agent is characterized by its solubility parameter, as defined by Vaughan in Cosmetics and Toiletries, Vol. 103, p. 47-69, October 1988 (expressly incorporated herein by reference). A lipophilic skin-moisturizing agent having a Vaughan solubility Parameter (VSP) from 5 to 10, preferably from 5.5 to 9 is suitable for use in antimicrobial cleansing embodiments of the inventive antimicrobial compositions.

A wide variety of “lipid-type materials” and mixtures of materials will be suitable for use in embodiments of antimicrobial compositions of the present invention. “Lipid-type materials” means lipophilic compounds, and include lipophilic skin conditioning agents. Some such skin conditioning agents are: hydrocarbon oils and waxes; silicones; fatty acid derivatives; cholesterol; cholesterol derivatives; di- and tri-glycerides; vegetable oils; vegetable oil derivatives; liquid nondigestible oils (such as those described in U.S. Pat. No. 3,600,186 to Mattson, issued Aug. 17, 1971 and U.S. Pat. Nos. 4,005,195 and 4,005,196 to Jandacek et al., both issued Jan. 25, 1977) all of which are herein incorporated by reference; or blends of liquid digestible or nondigestible oils with solid polyol polyesters (such as those described in U.S. Pat. No. 4,797,300 to Jandacek, issued Jan. 10, 1989; U.S. Pat. Nos. 5,306,514, 5,306,516 and 5,306,515 to Letton, all issued Apr. 26, 1994, all of which are herein incorporated by reference); and acetoglyceride esters; alkyl esters; alkenyl esters; lanolin and its derivatives; milk tri-glycerides; wax esters; beeswax derivatives; sterols; phospholipids; and mixtures of any or all of the foregoing. Fatty acids, fatty acid soaps and water soluble polyols are specifically excluded from this definition of a lipophilic skin moisturizing agent.

Some examples of lipid-type materials are: petrolatum; mineral oil microcrystalline waxes; polyalkenes (for example hydrogenated and nonhydrogenated polybutene and polydecene); paraffins; cerasin; ozokerite; polyethylene; and perhydrosqualene. Blends of petrolatum and hydrogenated and nonhydrogenated high molecular weight polybutenes, wherein the ratio of petrolatum to polybutene ranges from about 90:10 to about 40:60, are also suitable for use in some embodiments as the lipid skin moisturizing agent in the compositions herein.

Some additional examples of lipid-type materials are: dimethicone copolyol; dimethylpolysiloxane; diethylpolysiloxane; high molecular weight dimethicone; mixed C₁-C₃₀ alkyl polysiloxane; phenyl dimethicone; dimethiconol, and mixtures of any two or more of the foregoing. More preferred in some embodiments are non-volatile silicones selected from dimethicone; dimethiconol; mixed C1-C30 alkyl polysiloxane; and mixtures of any two or more thereof. Nonlimiting examples of silicones useful in some embodiments are described in U.S. Pat. No. 5,011,681, to Ciotti et al., issued Apr. 30, 1991, which is incorporated by reference.

Some additional examples of lipid-type materials are: castor oil; soy bean oil; derivatized soybean oils such as maleated soy bean oil; safflower oil; cotton seed oil; corn oil; walnut oil; peanut oil; olive oil; cod liver oil; almond oil; avocado oil; palm oil and sesame oil; vegetable oils and vegetable oil derivatives; coconut oil and derivatized coconut oil; cottonseed oil and derivatized cottonseed oil; jojoba oil; cocoa butter; and the like, as well as mixtures of any two or more thereof. Acetoglyceride esters are useful in some embodiments; and an example is acetylated monoglyceride. Lanolin and its derivatives are preferred in some embodiments; and some examples are: lanolin, lanolin oil, lanolin wax, lanolin alcohols, lanolin fatty acids, isopropyl lanolate, acetylated lanolin, acetylated lanolin alcohols, lanolin alcohol linoleate and lanolin alcohol riconoleate.

In some embodiments, it is most preferred that at least 75% of the lipophilic skin conditioning agent consists of lipids selected from the group consisting of: petrolatum; blends of petrolatum and high molecular weight polybutene; mineral oil; liquid nondigestible oils (for example liquid cottonseed sucrose octaesters); or blends of liquid digestible or nondigestible oils with solid polyol polyesters (for example sucrose octaesters prepared from C₂₂ fatty acids), wherein the ratio of liquid digestible or nondigestible oil to solid polyol polyester ranges from about 96:4 to about 80:20; hydrogenated or nonhydrogenated polybutene; microcrystalline wax; polyalkene; paraffin; cerasin; ozokerite; polyethylene; perhydrosqualene; dimethicones; alkyl siloxane; polymethylsiloxane; methylphenylpolysiloxane; and mixtures of any two or more thereof. In embodiments comprising a blend of petrolatum and other lipids, the ratio of petrolatum to the other selected lipids (hydrogenated or unhydrogenated polybutene or polydecene or mineral oil) is preferably from about 10:1 to about 1:2, more preferably from about 5:1 to about 1:1.

In some embodiments wherein a lipophilic skin moisturizing agent is employed as the mildness enhancer in the inventive antimicrobial compositions, a stabilizer will be included at a level ranging from about 0.1% to about 10%, preferably from about 0.1% to about 8%, more preferably from about 0.1% to about 5% by weight of the antimicrobial composition. A “stabilizer” is a compound or mixture that forms a crystalline stabilizing network in the liquid composition that prevents the lipophilic skin moisturizer agent droplets from coalescing and phase splitting in the product. The network exhibits time-dependent recovery of viscosity after shearing (for example, thixotropy).

The stabilizers disclosed above do not include surfactants. The stabilizers provide improved shelf- and stress-stability. In some embodiments, preferred hydroxyl-containing stabilizers include 12-hydroxystearic acid, 9,10-dihydroxystearic acid, tri-9,10-dihydroxystearin and tri-12-hydroxystearin (hydrogenated castor oil is mostly tri-12-hydroxystearin). Tri-12-hydroxystearin is most preferred in some embodiments of the inventive compositions. When these crystalline, hydroxyl-containing stabilizers are utilized in embodiments of the antimicrobial compositions herein (for example especially cleansing compositions), they are typically present at from about 0.1% to 10%, preferably from 0.1% to 8%, more preferably from 0.1% to about 5% of the antimicrobial compositions. The stabilizer is insoluble in pure water under ambient to near ambient conditions.

In some embodiments, the stabilizer employed in the antimicrobial compositions herein comprises a polymeric thickener. A “thickener” is a compound capable of increasing the viscosity of a liquid composition, but which don't necessarily form the aforementioned cross-linked matrix. Particular thickeners are described in more detail herein. When polymeric thickeners are used as stabilizers in embodiments of the inventive antimicrobial compositions, they are typically included in an amount ranging from about 0.01% to about 5%, preferably from about 0.3% to about 3%, by weight of the composition. In some embodiments, the polymeric thickener is preferably an anionic, nonionic, cationic or hydrophobically modified polymer selected from the group consisting of: cationic polysaccharides of the cationic guar gum class with molecular weights of 1,000 to 3,000,000; anionic, cationic, and nonionic homopolymers derived from acrylic and/or methacrylic acid; anionic, cationic, and nonionic cellulose resins; cationic copolymers of dimethyldialkylammonium chloride, and acrylic acid; cationic homopolymers of dimethylalkylammonium chloride; cationic polyalklene and ethoxypolyalkylene imines; polyethylene glycol of molecular weight from 100,000 to 4,000,000; and mixtures of two or more thereof. In some embodiments, the polymer is preferably selected from the group consisting of sodium polyacrylate, hydroxy ethyl cellulose, cetyl hydroxy ethyl cellulose, and polyquatemium 10.

In some embodiments, the stabilizer employed in the cleansing compositions will comprise C₁₀-C₂₂ ethylene glycol fatty acid esters. In some embodiments, C₁₀-C₂₂ ethylene glycol fatty acid esters are desirably combined with the polymeric thickeners (described above). In some embodiments, the ester is preferably a diester, more preferably a C₁₄-C₁₈ diester, most preferably ethylene glycol distearate. In embodiments wherein C₁₀-C₂₂ ethylene glycol fatty acid esters are utilized as the stabilizer, they will be present in a concentration range of: from about 3% to about 10%, preferably from about 5% to about 8%, more preferably from about 6% to about 8% of the personal cleansing compositions.

Another class of stabilizer which will be employed in some embodiments of the antimicrobial compositions of the present invention comprises dispersed amorphous silica: i.e. fumed silica, precipitated silica and mixtures thereof. As used herein the term “dispersed amorphous silica” refers to small, finely divided non-crystalline silica having a mean agglomerate particle size of less than about 100 microns.

In some embodiments in which amorphous silicas are used as the stabilizer, they will be included in the cleansing compositions at levels ranging from about 0.1% to about 10%, preferably from about 0.25% to about 8%, more preferably from about 0.5% to about 5%.

Another class of stabilizer which will be employed in embodiments of the antimicrobial compositions of the present invention comprises dispersed smectite clay selected from the group consisting of bentonite and hectorite and mixtures thereof. Bentonite is a colloidal aluminum clay sulfate. (See Merck Index, Eleventh Edition, 1989, entry 1062, p. 164, which is incorporated by reference.) Hectorite is a clay containing sodium, magnesium, lithium, silicon, oxygen, hydrogen and flourine. (See Merck Index, eleventh Edition, 1989, entry 4538, p. 729, which is herein incorporated by reference.) When smectite clay is employed as the stabilizer in some embodiments of the cleansing compositions of the present invention, it will be constitute about 0.1% to about 10%, preferably from about 0.25% to about 8%, more preferably from about 0.5% to about 5% of the composition.

Other known stabilizers, such as fatty acids and fatty alcohols, are also employed in some embodiment of the. inventive compositions. In some embodiments, palmitic acid and lauric acid are especially preferred.

Some embodiments of the antimicrobial compositions of the present invention comprise a wide range of optional ingredients. The CTFA International Cosmetic Ingredient Dictionary, Sixth Edition, 1995, which is incorporated by reference herein in its entirety, describes a wide variety of non-limiting cosmetic and pharmaceutical ingredients commonly used in the skin care industry, which are suitable for use in various embodiments of the compositions of the present invention. Non-limiting examples of functional classes of ingredients are described at page 537 of this reference, which is expressly incorporated herein by reference.

Examples of functional classes employed in various embodiments of the invention include: abrasives, anti-acne agents, anticaking agents, antioxidants, binders, biological additives, bulking agents, chelating agents, chemical additives, colorants, cosmetic astringents, cosmetic biocides, denaturants, drug astringents, emulsifiers, emollients, external analgesics, film formers, fragrance components, humectants, opacifying agents, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful in embodiments of the invention include solubilizing agents, sequestrants, and keratolytics, and the like.

A “colorant” is any compound or mixture capable of imparting a color to the composition.

An “emollient” is an compound or mixture capable of making the skin more soft or supple.

Some embodiments comprise one or more antioxidants, examples of which are: amino acids or amino acid derivatives; imidazoles and their derivatives; peptides such as D,L-carnosin; carotinoids; carotines and their derivatives; liponic acid; metal chelating agents (such as alpha-hydroxy fatty acids, palmitinic acid, phytinic acid, lactoferrine); alpha-hydroxyacids (for example lactic acid, maleic acid); humic acid; gallate; EDTA, EGTA and their derivatives; unsaturated fatty acids and their derivatives; vitamin C (ascorbic acid) and its derivatives (such as acetylated derivatives); rutinic acid and its derivatives; alpha-glycosyl rutin, ferulic acid, butylhydroxytoluol, butylhydroxyanisol and suitable derivatives; and/or mixtures of two or more of these substances.

In some embodiments, the inventive compositions comprise one or more UV absorbers, such as one or more of:
p-aminobenzoic acid and/or its derivatives, for example 4-dimethylaminobenzoic acid 2-ethylhexyl ester;
salicylic acid and/or its derivatives, for example salicylic acid 2-ethylhexyl ester;
benzophenone derivatives, for example 2-hydroxy-4-methoxybenzophenone and its 5-sulfonic acid derivative;
dibenzoylmethane derivatives, for example 1-(4-tert-butylphenyl)-3-(4-methoxyphenyl)propane-1,3-dione;diphenylacrylates, for example 2-ethylhexyl 2-cyano-3,3 -diphenylacrylate, and 3-(benzofuranyl) 2-cyanoacrylate;3-imidazol-4-ylacrylic acid and esters;
benzofuran derivatives, especially 2-(p-aminophenyl)benzofuran derivatives, described in EP-A-582 189, U.S. Pat. No. 5,338,539, U.S. Pat. No. 5,518,713 and EP-A-613 893;
polymeric UV absorbers, for example the benzylidene malonate derivatives described in EP-A-709 080;
cinnamic acid derivatives, for example the 4-methoxycinnamic acid 2-ethylhexyl ester and isoamyl ester or cinnamic acid derivatives described in U.S. Pat. No. 5,601,811 and WO 97/00851;
camphor derivatives, for example 3-(4′-methyl)benzylidene-bornan-2-one, 3-benzylidenebornan-2-one, N-[2(and 4)-2-oxyborn-3-ylidene-methyl)-benzyl]acrylamide polymer, 3-(4′-trimethylammonium)-benzylidene-bornan-2-one methyl sulfate, 3,3′-(1,4-phenylenedimethine)-bis(7,7-dimethyl-2-oxo-bicyclo[2.2.1]heptane-1-methanesulfonic acid) and salts, 3-(4′-sulfo)benzylidene-bornan-2-one and salts; camphorbenzalkonium methosulfate;
hydroxyphenyltriazine compounds, for example 2-(4′-methoxyphenyl)-4,6-bis(2′-hydroxy-4′-n-octyloxyphenyl)-1,3,5-triazine; 2,4-bis{[4-(3-(2-propyloxy)-2-hydroxy-propyloxy)-2-hydroxy]-phenyl}-6-(4-methoxyphenyl)-1,3,5-triazine; 2,4-bis{[4-(2-ethyl-hexyloxy)-2-hydroxy]-phenyl}-6-[4-(2-methoxyethyl-carboxyl)-phenylamino]-1,3,5-triazine; 2,4-bis{[4-(tris(trimethylsilyloxy-silylpropyloxy)-2-hydroxy]-phenyl}-6-(4-methoxyphenyl)-1,3,5-triazine; 2,4-bis{[4-(2″-methylpropenyloxy)-2-hydroxy]-phenyl}-6-(4-methoxyphenyl)-1,3,5-triazine; 2,4-bis{[4-(1′,1′,1′,3′,5′,5′,5′-heptamethyltrisilyl-2″-methyl-propyloxy)-2-hydroxy]-phenyl}-6-(4-methoxyphenyl)-1,3,5-triazine; 2,4-bis{[4-(3-(2-propyloxy)-2-hydroxy-propyloxy)-2-hydroxy]-phenyl}-6-[4-ethylcarboxy)-phenylamino]-1,3,5-triazine;
benzotriazole compounds, for example 2,2′-methylene-bis(6-(2H-benzotriazol-2-yl)-4-(1,1,3,3-tetramethylbutyl)-phenol;
trianilino-s-triazine derivatives, for example 2,4,6-trianiline-(p-carbo-2′-ethyl-1′-oxy)-1,3,5-triazine and the UV absorbers disclosed in U.S. Pat. No. 5,332,568, EP-A-517 104, EP-A-507 691, WO 93/17002 and EP-A-570 838;
2-phenylbenzimidazole-5-sulfonic acid and salts thereof;
menthyl o-aminobenzoates;
physical sunscreen agents coated or not as titanium dioxide, zinc oxide, iron oxides, mica, MnO, Fe₂O₃, Ce₂O₃, Al₂O₃, ZrO₂. (surface coatings: polymethylmethacrylate, methicone (methylhydrogenpolysiloxane as described in CAS 9004-73-3), dimethicone, isopropyl titanium triisostearate (as described in CAS 6141749-0), metal soaps as magnesium stearate (as described in CAS 4086-70-8), perfluoroalcohol phosphate as C9-15 fluoroalcohol phosphate (as described in CAS 74499-44-8; JP 5-86984 , JP 4-330007)). The primary particle size is an average of 15 nm-35 nm and the particle size in dispersion is in the range of 100 nm -300 nm;
aminohydroxy-benzophenone derivatives disclosed in DE 10011317, EP 1133980 and EP 1046391;
phenyl-benzimidazole derivatives as disclosed in EP 1167358;
the UV absorbers described in “Sunscreen agents”, Eds. N. J. Lowe, N. A. Shaath, Marcel Dekker, Inc. , New York and Basle or in Cosmetics & Toiletries (107), 50ff (1992) also can be used as additional UV protective substances.

Exemplary embodiments of the inventive compositions include: skin-care preparations; bath preparations; cosmetic personal care preparations such as facial care preparations and skin preparations; feminine hygiene and intimate care products; foot-care preparations; light-protective preparations; after sun care preparations; skin-tanning preparations; depigmenting preparations; insect-repellents; deodorants; antiperspirants; preparations for cleansing and caring for blemished skin; hair-removal preparations in chemical form (depilation); shaving preparations; oral care preparations; fragrance preparations and cosmetic hair-treatment preparations.

In various embodiments, the final formulation of inventive compositions take a wide variety of preparation forms, for example in the form of liquid preparations as a water-in-oil (W/O), oil-in-water (O/W), oil-in-water-in-oil (O/W/O), water-in-oil-in-water (W/O/W) or phase inversion transfer (PIT) emulsions and all kinds of microemulsions, in the form of a gel, an oil, a cream, milk or lotion, a powder, a lacquer, a tablet or make-up, a stick, a spray or an aerosol, a foam, a tonic, a surfactant, a liquid soap preparation, a bar soap preparation or a paste. Embodiments of the present invention include a wide variety of cosmetic preparations and pharmaceutical preparations that contain silver ion-containing aqueous acid of the present. invention (inventive silver dihydrogen citrate; silver dihydrogen citrate).

In various embodiments of the inventive composition, the composition is in the form of an emulsion. In such inventive embodiments, emulsifiers will be used, such as: carboxylic acids and their salts (such as palmitinic acid, stearic acid, oleic acid, lauric acid etc.); alkyl phosphates or phosphoric acid esters (such as diethanolamine cetyl phosphate, potassium cetyl phosphate, etc.); alkylamines; alkyl imidazolines; ethoxylated amines; quaternary emulsifiers; sorbitol and sorbitan (polysorbates, sorbitan esters); sucrose and glucose derivatives (such as sorbitan stearate, sucrose cocoate, methyl glucose-sesquistearate, methyl glucose dioleate and methyl glucose isostearate); alkanolamides and ethoxylated amides (such as PEG-n acylamides (with n=1-50)); ethoxylated carboxylic acids or polyethylene glycol esters (PEG-n acylates with n=1-200), such as fatty alcohol; polyglycolethers; laureth-n (with n=1-200); ceteareth-n (with n=1-200); steareth-n (with n=1-100); oleth-n (with n=1-200) and PEG-n stearate (with n=1-200); PEG-n oleate (with n=1-200); PEG-n cocoate (with n =2-150); polyglyceryl esters and fatty acid esters; dimethicone copolyols such as silicone polyethylene oxide copolymer; silicone glycol copolymer; propoxylated or polyoxyethylene ethers; polaxamers; polymeric emulsifiers (such as acrylate copolymers or crosspolymers and acrylamides or polyacrylamides); and mixtures or combinations of two or more of the foregoing emulsifiers.

In emulsified embodiments of the present invention, the lipid phase will advantageously be selected from mineral oils; mineral waxes; oils (such as triglycerides of capric and caprylic acid); natural oils (such as castor oil); fats; waxes and other natural and synthetic fats (for example esters of fatty acids with short chain alcohols, such as isopropanol, propylene glycol or glycerine) or esters of fatty alcohols with fatty acids or carboxylic acids with low number of carbon atoms; alkylbenzoate; silicone oils (such as dimethylpolysiloxane, diethylpolysiloxane, diphenylpolysiloxane); and/or mixtures of two or more thereof.

In various embodiments of the present invention, the oil phase of the emulsion, oleogel, hydrodispersion or lipodispersion is advantageously selected from saturated and/or unsaturated, branched and/or linear alkane carbonic acids with a chain length of 3 to 30 C-atoms; saturated and/or unsaturated, branched or linear alcohols with a chain length of 3 to 30 C-atoms; an ester of aromatic carbonic acids and saturated and /or unsaturated, branched and/or linear alcohols with a chain length of 3-30 C-atoms; and/or mixtures of two or more thereof.

In some embodiments, exemplary ester oils are: isopropylmyristate, isopropylpalmitate, isopropylstearate, isopropyloleate, n-butylstearate, n-hexyllaurate, n-decyloleate, isooctylstearate, iso-nonylstearate, isononylisononanoate, 2-ethylhexylpalmitate, 2-hexyllaurate, 2-hexyldecylstearate, 2-octyldodecylpalmitate, oleyloleate, oleylerucate, erucyloleate and erucylerucate, as well as synthetic, semi-synthetic and natural mixtures of such esters such as jojoba oil.

In some embodiments comprising fatty acid triglycerides, they will be selected from synthetic, semi-synthetic and natural oils, such as: olive oil, sunflower oil, soy oil, peanut oil, rape-seed oil, palm oil, almond oil, coconut oil and similar oils.

Mixtures of such oil and wax components or waxes such as cetyl palmitate will be used in some embodiments as the sole oil phase.

In some embodiments, the oil phase comprises other preferred ingredients, such as: 2-ethylhexylisostearate; octyldodecanol; isotridecylisononanoate; isoeicosane; 2-ethylhexylcocoate; C₁₂-C₁₅ alkyl benzoate; caprylic-caprinic acid-triglycerides and dicaprylic ether or mixtures of those ingredients (such as mixtures of 2-ethylhexylisostearate with C₁₂-C₁₅ alkylbenzoate); mixtures of C₁₂-C₁₅ alkylbenozoate and isotridecylisononanoate and mixtures of C₁₂-C₁₅ alkylbenzoate with 2-ethylhexylisostearate and isotridecylisononanoate. Moreover cyclic or linear silicone oils can be used and are in some cases the only ingredient in the oil phase. In particular embodiments, preferred silicone oils include: cyclomethicone (octamethylcyclotetrasiloxane), hexamethylcyclotrisiloxane, polydimethylsiloxane and poly(methylphenylsiloxane).

In some embodiments, preferred hydrocarbons include: paraffin oil, squalane and squalene.

In some embodiments, the aqueous phase contains for example ingredients such as: alcohols, diols or polyols with a low number of C-atoms or their ethers (for example ethanol, isopropanol, propyleneglycol, glycerin, ethylene glycol, ethylene glycol monoethylether, ethylene glycol monobutylether, propylene glycol monomethylether, propylene glycol monoethylether, propylene glycol monobutylether, diethylene glycol monomethylether; diethylene glycol monoethylether, diethylene glycol monobutylether and similar products); lower homologs of alcohols (such as ethanol, isopropanol, 1,2-dipropandiol and glycerin), as well as one or more thickeners for example: silicium dioxide, aluminum silicates, polysaccharides or derivatives thereof (for example hyaluronic acid, xanthan gum, hydroxypropylmethylcellulose); polyacrylates {for example substances from the Carbopol range (for example Carbopol types 980, 981, ETD2001 or 2020, Aqua SF-1, Ultrez 1 ), Salcare range (Salcare SC80, Salcare SC81, Salcare SC91, Salcare AST, Salcare SC 92, Salcare SC95, Salcare SC96, Salcare Super 7) or Novemer™ EC-1}; Cosmedia® SP; Aristoflex AVC; or modified Starch (such as Structure® Solanace or Structure®XL).

The invention further provides personal care compositions, which are oral care compositions, comprising silver dihydrogen citrate and water in an orally acceptable form. By “orally acceptable form”, it is meant that the oral care composition includes at least one ingredient other than silver dihydrogen citrate, an alcohol, a detergent or combinations thereof, and that the ingredient is of the type that is tolerated by teeth and buccal tissues, such as the gums and inner cheek. Such orally acceptable compositions need not be ingestible (as most fluoride-containing toothpastes are not considered ingestible due their fluoride content), are non-toxic when applied to the mouth and then removed from the mouth. In particular, the invention provides oral care compositions that are mouth rinses, mouth washes, tooth pastes, tooth gels, denture pastes, denture gels, chewing gums, solid lozenges and oral sprays, which are described in more detail herein.

In some embodiments, the oral care compositions contain one or more additional oral care ingredients for treating the mouth, including the teeth, gums, tongue, or buccal skin surfaces. Such additional ingredients include cleaning agents, abrasives, fluoridating agents, malodor treating agents, tooth whitening agents, anti-carries agents, gelling agents, antibacterial agents (other than the inventive antimicrobial agent), flavorings, colorants and combinations of two or more of the foregoing. Such oral compositions may be used in a conventional manner commensurate with the physical form of the compositions, which may be liquid, paste, semi-solid or solid. For example, in some embodiments, wherein the compositions are pastes or gels, they are applied to a mouth surface (for example teeth and/or gums) with brushing. In other embodiments, where the compositions are liquids, they are applied to the mouth surface with gargling or swishing. They may be removed from the mouth by expectorating and optionally rinsing with water or a mouth rinse.

The invention provides antimicrobial compositions that possess antimicrobial activity against oral bacteria, and thus exhibit antibacterial effects in oral care applications. In particular embodiments, inventive compositions fight plaque; reduce, slow the progression of, or prevent gingivitis; reduce, slow the progression of, or prevent periodontitis and/or reduce mouth malodor. Such oral antimicrobial activity is enhanced in some inventive embodiments by: combining the silver dihydrogen citrate with other antimicrobial, anti-plaque, anti-gingivitis and/or anti-periodontitis agents such as chlorhexidine salts, quaternary compounds (such as cetrimonium bromide, benzalkonium chloride and cetyl pyridinium chloride) and/or phenolic substances {such as 2,4,4′-trichloro-2′-hydroxydiphenylether; 4,4′-dichloro-2-hydroxydiphenylether, thymol, and other phenolic compounds having the following generic formula 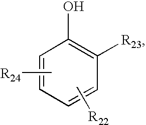
wherein R₂₂, R₂₃ and R₂₄ are independently from each other alkyl (branched, cyclo or linear), aryl, O-aryl, o-alkyl (linear, cyclo, or branched)}.

The invention further provides anti-plaque, anti-gingivitis and/or anti-periodontitis agents are for example thymol; 2-t-butyl-5-(4-t-butylphenyl)-phenol; 2,4-di-t-butyl phenol; 2-cyclohexylmethyl-4-t-butylphenol; 2-t-octyl-5-cyclohexylmethylphenol; 2-t-butyl-4-(1,1-dimethylpropyl)phenol; 2-t-butyl-4-(1,1-dimethylbutyl)phenol; 2,4-di-t-butyl-5-methylphenol; 2-t-butyl-4-(1,1,2,2-tetramethylpropyl)-5-methylphenol; 2-t-butyl-4-( 1,1,2,2-tetramethylpropyl)-phenol; 2-t-butyl-5-cyclohexylmethylphenol; 2-t-butyl-4-n-heptylphenol; 2-isopropyl-5-cyclo-hexylmethylphenol; 2-isopropyl-4-cyclohexylmethylphenol; and 2-cyclohexyl-4-n-heptyl-phenol.

In some embodiments, the invention provides oral care compositions containing the silver dihydrogen citrate alone, or in combinations with one or more of the above mentioned antimicrobial and/or anti-plaque agents are for example mouth rinses, semi-solids such as toothpastes or gel dentifrices, chewing gums or solid lozenge or the like.

Further embodiments of inventive oral compositions contain, for example:
polishing agents (such as silica gels, colloidal silica or complex amorphous alkali metal aluminosilicate, sodium bicarbonate, sodium metaphosphate, potassium metaphosphate, tricalcium phosphate, dehydrated dicalcium phosphate, anhydrous dicalcium phosphate, calcium pyrophosphate, calcium carbonate, aluminum silicate, hydrated alumina, silica, bentonite and mixtures of any two or more thereof);
humectants (such as glycerin, sorbitol, an alkylene glycol such as polyethylene glycol or propylene glycol and/or mixtures of any two or more thereof);
water (for example as hereinbefore described);
natural or synthetic thickener or gelling (agent such as Irish moss, iota-carragenan, kappa-carrageenan, gum tragacanth, starch, polyvinylpyrrolidone, hydroxyethyl propyl cellulose, hdroxybutyl methyl cellulose, hydroxypropyl methyl cellulose, hydroxyethyl cellulose and sodium carboxymethyl cellulose);
alcohol (such as ethanol or isopropanol);
organic surface-active agents, which are cationic, anionic or non-ionic;
flavoring agents (such as thymol, menthol, methyl salicylate (wintergreen oil), eucalyptol, carvacrol, camphor, anethole, carvone, eugenol, isoeugenol, limonene, losimen, n-decyl alcohol, citronel, a-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, gualacol, lavender oil, mustard oil, phenol, phenyl salicylate, pine oil, pine needle oil, sassafras oil, spike lavender oil, storax, thyme oil, tolu balsam, terpentine oil, clove oil and combinations of two or more thereof; some preferred flavoring oils are: for example oil of spearmint, peppermint, wintergreen, sassafras, clove, sage, eucalyptus, cinnamon, lemon, orange and methyl salicylate);
sweetening agents (such as sucrose, lactose, maltose, xylitol, sodium cyclamate, perillartine, aspartyl phenyl alanine methyl ester, saccharine and the like);
agents used to diminish teeth sensitivity (such as strontium chloride, potassium nitrate and potassium citrate);
whitening agents (for example peroxides, such as urea peroxide, carbamide peroxide and/or hydrogen peroxide);
preservatives (such as sodium benzoate);
substances that release fluoride ions to protect against caries (such as inorganic fluoride salts, for example sodium, potassium, ammonium or calcium fluoride or organic fluorides such as amine fluoride);
other agents (such as chlorophyll compounds) and/or ammoniated materials (such as urea, diammonium phosphate) and/or mixtures thereof;
The invention further provides oral care compositions comprising antibacterial enhancing agents. Such “antibacterial enhancing agents” contain a delivery enhancing group, which attaches or substantively, adhesively, cohesively or otherwise bonds the antibacterial enhancing agents with the antibacterial and/or anti-plaque agent to the oral (for example tooth and gum) surface, and a retention-enhancing group (generally a hydrophobic group), which attaches or otherwise bonds the antimicrobial and/or anti-plaque agent to the antibacterial enhancing agent. These substances thus deliver the antimicrobial and/or anti-plaque agent to the tooth and/or gum surface, and promote retention of the active on the surface, which improves the retardation of plaque growth on oral surfaces.

In some embodiments, the antibacterial enhancing agent is an anionic polymer comprising a chain or backbone containing repeating units each preferably containing at least one carbon atom and preferably at least one directly or indirectly pendent, monovalent delivery-enhancing group and at least one directly or indirectly pendent monovalent retention-enhancing group generally, vicinally or less preferable otherwise bonded to atoms, preferably carbon, in the chain.

In some embodiments, the antibacterial enhancing agent may be a simple compound, preferably a polymerizable monomer, more preferably a polymer or mixture of two or more polymers such as: oligomers, homopolymers, copolymers of two or more monomers, ionomers, block copolymers, graft polymers, cross-linked polymers and copolymers, and the like. The antibacterial enhancing agent may be: natural or synthetic; water soluble (for example saliva) soluble or swellable (hydratable, hydrogel forming); and having an (weight) average molecular weight of about 100 to about 5,000,000, preferably about 1,000 to about 1,000,000, more preferably about 25,000 to 500,000.

In some embodiments comprising polymeric antibacterial enhancing agents, it is desirable for maximizing delivery and retention of the antimicrobial and/or anti-plaque agent to oral surfaces, that the repeating units in the polymer chain or back-bone containing the acidic delivery enhancing groups constitute at least about 10%, preferably at least about 50%, more preferably at least about 80% up to 95% or 100% by weight of the polymer.

In some embodiments, the antibacterial enhancing agent will contain at least one delivery-enhancing group, which is preferably acidic (such as sulfonic, phosphinic, or more preferably phosphonic or carboxylic) or a salt thereof, for example alkali metal or ammonium; and at least one organic retention-enhancing group (such typically groups having the formula —(X)ₙ—R₂₃ wherein X is O, N, S, SO, SO₂, P, PO or Si or the like, R₂₃ is hydrophobic alkyl, alkenyl, acyl, aryl, alkaryl, aralkyl, heterocyclic or their inert-substituted derivatives, and n is zero or 1 or more).

In some embodiments, the aforesaid “inert-substituted derivatives” include substituents on R₂₃ that are non-hydrophilic and do not significantly interfere with the desired function of the antibacterial enhancing agent as enhancing the delivery of the antimicrobial and/or anti-plaque agent to and retention thereof on oral surfaces such as halo, for example Cl, Br, I, and carbocyclyl and the like. In some embodiments, the antibacterial enhancing agent is preferably a natural or synthetic anionic polymeric carboxylate having a molecular weight of about 1,000 to about 5,000,000 preferably about 30,000 to about 500,000.

The invention further provides home care compositions comprising silver dihydrogen citrate in a laundry detergent and/or fabric care composition. In such embodiments of the invention, the inventive laundry detergent and/or fabric care compositions preferably further comprise a detergent ingredient selected from cationic, anionic and/or nonionic surfactants and/or bleaching agent.

In some embodiments, the antimicrobial laundry detergent and/or fabric care compositions according to the invention can be liquid, paste, gels, bars, tablets, spray, foam, powder or granular forms. Granular compositions can also be in “compact” form, the liquid compositions can also be in a “concentrated” form.

In some embodiments, compositions of the invention may for example, be formulated as hand and machine laundry detergent compositions including laundry additive compositions and compositions suitable for use in the soaking and/or pretreatment of stained fabrics, rinse added fabric softener compositions. Pre-or post treatment of fabric include gel, spray and liquid fabric care compositions. A rinse cycle with or without the presence of softening agents is also contemplated.

When formulated as compositions suitable for use in a laundry machine washing method, embodiments of the compositions of the invention preferably contain both a surfactant and a builder compound; and additionally one or more detergent components, such as: organic: polymeric compounds; bleaching agents; additional enzymes; suds suppressors; dispersants; lime-soap dispersants; soil suspension and anti-redeposition agents; and corrosion inhibitors. Some embodiments of the inventive laundry compositions also contain softening agents as additional detergent components.

Some embodiments of the invention that are laundry detergent and/or fabric care compositions optionally further contain cationic fabric softening components, which include: water-insoluble quatemary-ammonium fabric softening actives (or the corresponding amine precursor), the most commonly used being di-long alkyl chain ammonium chloride or methyl sulfate.

In exemplary embodiments, preferred cationic softeners are ditallow dimethylammonium chloride (DTDMAC); dihydrogenated tallow dimethylammonium chloride; dihydrogenated tallow dimethylammonium methylsulfate; distearyl dimethylammonium chloride; dioleyl dimethylammonium chloride; dipalmityl hydroxyethyl methylammonium chloride; stearyl benzyl dimethylammonium chloride; tallow trimethylammonium chloride; hydrogenated tallow trimethylammonium chloride; C₁₂-₁₄alkyl hydroxyethyl dimethylammonium chloride; C₁₂-₁₈. alkyl dihydroxyethyl methylammonium chloride; di-(stearoyloxyethyl) dimethylammonium chloride (DSOEDMAC); di-(tallow-oxy-ethyl) dimethylammonium chloride; ditallow imidazolinium methylsulfate; 1-(2-tallowylamidoethyl)-2-tallowyl imidazolinium methylsulfate; and/or mixtures or combinations of any two or more thereof.

Some laundry detergent and/or fabric care embodiments of the present invention may also contain ampholytic (i.e. amphoteric), zwitterionic, and semi-polar surfactants.

In some embodiments, the inventive laundry detergent and/or fabric care compositions will contain one or more enzymes that provide cleaning performance, fabric care and/or sanitization benefits. Examples of such enzymes include: cellulases, hemicellulases, peroxidases, proteases, gluco-amylases, amylases, xylanases, lipases, phospholipases, esterases, cutinases, pectinases, keratanases, reductases, oxidases, phenoloxidases, lipoxygenases, ligninases, pullulanases, tannases, pentosanases, malanases, -glucanases, arabinosidases, hyaluronidase, chondroitinase, laccase, and/or combinations or mixtures of any two or more thereof.

The invention further provides silver dihydrogen citrate laundry detergent compositions comprising a builder system. A conventional builder system is suitable for use in the inventive compositions. Suitable builder systems include: aluminosilicate materials silicates; polycarboxylates; alkyl- or alkenyl-succinic acid and fatty acids; chelating materials (such as ethylenediaminetetraacetate salts and diethylenetriaminepentamethyleneacetate salts); metal ion sequestrants (such as aminopolyphosphonates, particularly ethylenediamine tetramethylene phosphonic acid and diethylene triamine pentamethylenephosphonic acid); and mixtures and combinations of any two or more thereof. In some embodiments, the inventive compositions comprise one or more phosphate builders, alone or in combination with other builders.

In some embodiments, the antimicrobial laundry detergent and/or fabric care compositions herein also optionally contain one or more iron and/or manganese chelating agents. Such chelating agents will generally be selected from: amino carboxylates, amino phosphonates, polyfunctionally-substituted aromatic chelating agents and/or mixtures of any two or more thereof.

In some embodiments, the compositions contain water-soluble methyl glycine diacetic acid (MGDA) salts (or acid form) as a chelant or co-builder useful with, for example, insoluble builders such as zeolites, layered silicates and the like.

Another optional ingredient in some embodiments of the invention is a suds suppressor, exemplified by silicones, and silica-silicone mixtures.

Some embodiments of the invention include other components, such as: soil-suspending agents, soil-release agents, optical brighteners, abrasives, bactericides, tarnish inhibitors, coloring agents, and/or encapsulated or non-encapsulated perfumes may be employed.

An “abrasive” is a solid particulate compound or mixture which, through mechanical action, is capable of shearing residue from a surface. Abrasives are commonly found in oral compositions (such as tooth pastes), in facial cleansers, and hard surface cleansers.

Some embodiments of the invention that are laundry detergent and/or fabric care compositions invention also contain dispersants, such as water-soluble organic salts (for example homo- or co-polymeric acids or their salts, in which the polycarboxylic acid comprises at least two carboxyl radicals separated from each other by not more than two carbon atoms).

In some embodiments, the laundry detergent and/or fabric care compositions of the present invention include compounds for inhibiting dye transfer from one fabric to another of solubilized and suspended dyes encountered during fabric laundering operations involving colored fabrics.

In some embodiments, compositions according to the present invention are conveniently prepared as fluids. In some embodiments, the fluid is an aqueous liquid comprising silver dihydrogen citrate, water and citric acid, as well as additional water-soluble additives as described herein. Exemplary aqueous liquid compositions according to the present invention include: liquid soaps and/or detergents; liquid cleaning agents comprising one or more additional water-soluble additives, such as surfactants, water softeners and/or antibacterial agents (for example essential oils, alcohols, and others as mentioned herein); liquid oral compositions (for example mouth washes, optionally comprising one or more additional antimicrobial and/or flavoring agents, fluoridating agents, tooth whitening agents, anti-gingivitis and/or anti-periodontitis agents), liquid eyeglass or contact lens cleaning agents, liquid antiperspirant, deodorant or combined antiperspirant/deodorant compositions (optionally packaged as aerosols or roll-ons).

In some embodiments, compositions according to the present invention are dispersions, such as emulsions (liquid in liquid dispersions), colloidal suspensions (solid in liquid dispersions), foams (air in fluid suspensions), aerosols (liquid in air dispersions), etc. Emulsions include lotions, creams, milks, etc. In some embodiments, the silver dihydrogen citrate (i.e. silver ion in aqueous organic acid) forms the continuous phase of a fluid dispersion. For example, in some embodiments the inventive silver dihydrogen citrate of the present invention forms the continuous water phase of an oil-in-water emulsion (O/W), while the dispersed oil phase comprises one or more water-immiscible components. In such embodiments, it is generally necessary for the composition to include at least one emulsifier, as described in more detail herein, to retain droplets of dispersed oil phase stably suspended in the continuous water phase. Compositions of this type include lotions, creams, milks, microemulsions, etc.

In other fluid suspension embodiments, the silver dihydrogen citrate forms or is an ingredient in the liquid phase of a colloidal suspension. The dispersed phase comprises solid particles suspended in the continuous liquid phase. It is conventional to use a dispersing agent to maintain the solid particles in suspension.

In other fluid suspension embodiments, the silver dihydrogen citrate forms or is an ingredient of the dispersed phase of an emulsion. For example, in some embodiments, the composition is a water-in-oil emulsion (W/O), in which the silver dihydrogen citrate of the present invention is the dispersed (water) phase. In such embodiments, it is generally necessary to employ an emulsifier to maintain the dispersed water phase in suspension. As used here, the term “oil phase” means a relatively apolar phase that is immiscible in the water phase. Suitable oil phase components are not limited to oils per se, and are discussed in greater detail herein. Exemplary embodiments of such water-in-oil suspensions include medicinal oils and petrolatums (especially those containing one or more essential oils, especially camphor, menthol or mixtures thereof), creams, salves, etc.

The invention further provides “phase inversion temperature” (“PIT”) emulsions comprising silver dihydrogen citrate. The terms “phase inversion temperature emulsion” refers to an emulsion made by the phase inversion temperature (PIT) method. Aqueous silver dihydrogen citrate forms the continuous phase of a phase inversion temperature (PIT) type emulsion. In such embodiments, the oil and water phases are combined at a temperature above the phase transition temperature, or are combined and then heated to a temperature above the phase transition temperature. The phase transition temperature is the temperature at which the solution transitions from a an oil-in-water (O/W) to a water-in-oil (W/O) type of emulsion. The transition from O/W to W/O can be detected by observing one or more physical characteristics that are associated with the two different physical states. For example, a W/O composition has relatively poor conductance, whereas an O/W composition will have relatively high conductance. Also, a W/O composition will be readily diluted by oil, but not water, whereas an O/W composition will be readily diluted by water, but not oil. Additionally, a W/O composition will be evenly dyed by an oil-soluble, but not a water-soluble dye; an O/W composition will likewise be evenly dyed by a water-soluble, but not an oil-soluble dye.

Once the composition has formed the high-temperature W/O emulsion, the emulsion is cooled. At a temperature below the phase transition temperature, the suspension will transition from W/O to a stable O/W emulsion—generally without agitation. It is generally necessary to use an emulsifier or two or more co-emulsifiers in the PIT emulsion composition. Formation of a PIT emulsion requires use of an appropriate emulsifiers or combination of two or more co-emulsifiers, which are known in the art.

The invention provides silver dihydrogen citrate compositions in the form of a microemulsion. The term “microemulsion” applies to an emulsion in which the emulsion is generally transparent, the dispersed (oil) phase forming droplets that are effectively small enough that the emulsion does not substantially diffract visible light.

In some embodiments, the inventive silver dihydrogen citrate forms both the continuous phase and a layer of the dispersed phase of an emulsion. In such W/O/W embodiments, the dispersed phase comprises an oil phase that envelops a water phase layer, and the dispersed phase is suspended in the continuous phase. In such embodiments, it is necessary to use an emulsifier or a combination of two or more co-emulsifiers. Suitable emulsifiers are generally known in the art, and are described in some detail herein.

In some embodiments, both the continuous oil phase and an oil layer of the dispersed phase comprise oil; another layer (the outer layer) of the discrete phase is formed by the inventive silver dihydrogen citrate. It is necessary to use an emulsifier or combination of emulsifiers in such embodiments. Suitable emulsifiers are generally known in the art, and are described in some detail herein.

In some embodiments, the inventive antibacterial active is the water phase of a liposomal composition. Liposomes are small spherules of lipid layers encapsulating water layers. In some embodiments, the liposome consists of a single lipid bilayer encapsulating a water core. In other embodiments, the liposome consists of multiple lipid bilayers encapsulating multiple water layers. In general, a distribution of liposomes having various numbers of alternating lipid bilayers and water layers will be formed in the general process of making liposomes, which generally comprises drying a lipid composition (comprising one or more lipids) that will form the lipid layer(s) and then adding the water phase to the dried lipid composition with agitation. Other methods of making liposomes are known, and the inventive liposomal compositions are capable of being made by such other processes.

In some embodiments, the inventive antibacterial active forms the continuous phase of a liposomal composition, wherein the liposomes are suspended in the inventive antibacterial active. (Liposome suspension). In other embodiments, the inventive antibacterial active provides the fluid medium for a paste or cream comprising the liposomes. In particular embodiments, the polar layers of the liposomes optionally comprise the inventive antibacterial active. In some embodiments of the invention, liposomes are used in skin-treating compositions as antibacterials, antifungals and/or antivirals.

In other fluid compositions according to the present invention, the inventive silver dihydrogen citrate forms the liquid phase of a paste. In such embodiments, the paste comprises a discontinuous solid phase comprising at least one solid component that is insoluble in the inventive silver dihydrogen citrate. Exemplary embodiments of pastes according to the invention include: antibacterial medicinal pastes; tooth pastes (optionally including one or more of the following: fluoridating, flavoring, abrasive, whitening agents or combinations of two or more thereof), surface polishes (such as metal polishes, especially silver polishes); and the like.

In some embodiments, the silver dihydrogen citrate is combined with one or more gelling agents, such as water soluble polymers, crosslinked polymers, block copolymers or mixtures of polymers, to form a gel. “Gelling agents” are compounds capable of forming a cross-linked matrix within the water solvent. The silver dihydrogen citrate and water fill the interstices of the matrix. Depending on the degree of crosslinking and the amount of water solvent used in relation to the amount of gelling agent, the resulting gel composition will have a consistency from a free-flowing but viscous liquid, to a viscous fluid, to a semi-solid, to a solid of varying hardness. Such compositions may be used in personal care compositions (for example viscous fluid skin care gels; semisolid skin care gels; viscous fluid hair treatments; dental gels (optionally comprising one or more fluoridating agents, whitening agents, abrasive agents, and mixtures of two or more thereof); roll-on, glide-on or stick antiperspirants, deodorants or combined antiperspirant/deodorants; gel or semi-solid cuticle treatments; etc. As in other embodiments of the invention, the silver dihydrogen citrate may act as a preservative, as an active for treating a person to whom it is applied, or both. In particular, the invention provides gels having an opacifying agent added thereto, especially deodorant and antiperspirant gels.

In some embodiments, gels comprising the silver dihydrogen citrate according to the invention are used in various manufactured articles. In such embodiments, the gels are conveniently prepared in solid, semi-solid or viscous fluid form, depending on the use to which the articles are to be put. For example, in some embodiments according to the invention, the antimicrobial composition may be combined with known polymers, copolymers, block copolymers, or mixtures of thereof, to form solids, which optionally comprise one or more solid components dispersed therein, and/or optionally comprise one or more scenting agents, perfumes or essential oils as odor-enhancing agents. Such articles are, in some embodiments, formed into room deodorizers.

In embodiments where the gel phase is a viscous fluid, the inventive compositions may be used in: lip balms; protective coatings (for example waterproofing and antidesiccant coatings ); skin treatments (including medicinals, especially for use in wound treatment); buccal treatments (especially tooth brighteners and antibacterial dentrifice gels); skin protectants (including anti-chafing agents and sun blocks); fabric treatments (for example spot cleansers); and surface cleaners (for example metal cleaners).

In embodiments wherein the gel phase is a semi-solid, the inventive compositions are conveniently formed into: stick antiperspirants, deodorants or antiperspirant/deodorants; semi-solid room deodorizers, lip sticks, insect repellents, insect-bite treatments, wound treatments (for example antibiotic treatments); fabric treatments (for example spot cleansers).

In many embodiments, antimicrobial compositions of the invention are conveniently packaged in a form suitable for the intended use. Embodiments in which the inventive compositions are liquids are conveniently packaged: as aerosol sprays (generally in a container comprising a conventional propellant under pressure); pump sprays (for example in a container comprising a pump sprayer); as squirtable or pourable liquids; as douches, etc. In some embodiments, the inventive compositions are applied to pre-moistened articles (for example towelettes, sponges or abrasive pads), which are optionally packaged in a dispenser or individually in sealed pouches. In some embodiments, such articles are conveniently used to wipe down surfaces, such as: glass; appliances; ceramic bath fixtures; etc. In some embodiments such articles are conveniently used to clean skin, especially wounded skin and/or the skin of those who are sensitive to other antimicrobial agents.

Embodiments in which the inventive compositions are viscous fluids are conveniently packaged in tubes, squeezable bottles, jars or pots. In some embodiments, the viscous fluids are conveniently incorporated into articles that aid in their application to surfaces, as discussed with respect to liquid embodiments above.

Embodiments in which the inventive compositions are semi-solids are conveniently packaged in stick applicators (for example in deodorant, spot-cleaning, wound-treating, insect-bite treating, lip sticks, etc.), bars (for example soaps or detergents), or where the mode of action is by exposure to air, in a form which exposes the composition to the air, such as perforated packages, candle holders. In particular embodiments, semi-solid air-treating compositions are packaged in containers that may be reversibly opened and closed, and in particular embodiments, in containers that may be gradually opened to expose varying proportions of the composition's surface area to air.

Embodiments in which the inventive compositions are solid are conveniently packaged in stick applicators, or where the mode of action is by exposure to air, in a form which exposes the composition to the air, such as perforated packages, candle holders. In particular embodiments, solid air-treating compositions are packaged in containers that may be reversibly opened and closed, and in particular embodiments, in containers that may be gradually opened to expose varying proportions of the composition's surface area to air.

In the following non-limiting and illustrative examples, there are set forth formulations that exemplify particular embodiments of the present invention. The person of skill in the art will recognize that other embodiments of the invention are available using the description herein, and are contemplated as being within the scope of the present invention.

Personal care compositions in the following tables are specific embodiments of the present invention. In each of the compositions, the “silver dihydrogen citrate stock solution” is a solution of silver dihydrogen citrate and water, as herein described. In some embodiments the silver dihydrogen citrate stock solution comprises at least about 100 ppm, especially at least about 1,000 ppm, and more particularly at least about 2,000 ppm silver ion.

Some personal care products are applied to a person's body and left on the person's body. (It is to be expected that such products will eventually erode from the body or be washed off in the normal course of personal hygiene.) For example, when compositions according to the invention are prepared as wound-healing (for example antibacterial); skin treatment (for example moisturizing, protectant, etc.); anti-acne, anti-vaginitis, anti-dermatitis, insect repellant; and cosmetic compositions, they are commonly applied to the appropriate body surface and left to perform their desired function.

Thus, the invention provides methods of using personal care products for the treatment of body parts to achieve a beneficial effect. Such beneficial effects include the cleansing of body parts, the treatment of various conditions of the epithelial surfaces of body parts. Thus the methods provide for treatment of: the hair and scalp, for example to cleanse hair, to treat maladies of the scalp such as dandruff, etc.; the epidermis, for example to ameliorate or prevent dry skin, to treat acne, to protect the skin, to cleanse the skin, etc.; wounds, for example as a cleanser and to wound promote healing; vaginal tissues, for example to promote feminine hygiene, to relieve vaginitis, etc.; rectal tissue, for example as a cleanser, to relieve inflammation, to reduce irritation, etc; buccal tissues, for example to treat or prevent dental carries, to treat mouth ulcers, to reduce bacterial infestation in the mouth, etc.

Home care compositions that perform a cleansing function are commonly applied to an object (for example a fabric or hard surface) to be cleaned, and are then removed, for example with rinsing, wiping or scrubbing. For example, fabric treatments (for example fabric detergents) are commonly applied to a fabric (for example by rubbing a solid gel directly on the fabric, by spraying a liquid onto the fabric, or by adding a liquid to a water composition in which the fabric is agitated) and then removed, for example by rinsing with water. Surface treatments are commonly applied to the surface to be treated (for example glass, metal, tile or polymer surface), optionally agitated (for example with a mop or wipe) and then removed (for example by wiping or rinsing with water). In some embodiments, the surface treatment may be incorporated into an application means, for example a cloth or a pad, which is used not only to apply the surface treatment to the desired surface, but also to wipe it away. In particular embodiments, the surface treatment is applied to a pre-moistened wipe or pad, which may be optionally individually wrapped in a disposable package, or alternatively packaged in a re-sealable package, such as a resealable bag, box or pop-up dispenser.

Other home care compositions can be used as appropriate. For example, home care compositions used as air deodorizers are sprayed from a suitable sprayer (conveniently an aerosol spray can, which generally will contain one or more propellants); or are merely opened to the environment (for example as solid or semisolid gel deodorizers) to evaporate over time, thereby releasing deodorant into the ambient air. As another example, home care compositions used as surface treatments may be wiped onto a suitable surface and left to evaporate. Some preferred embodiments of the invention, however, exclude paints.

In the foregoing description, an adjective used to modify the term “agent” means a compound or mixture having the properties of, or capable of performing the function implied by the modifier. Such terms have the meanings conventionally recognized by one of skill in the art for such compounds and mixtures.

### EXAMPLE 1.

Preparation of Silver Dihydrogen Citrate

Water was introduced into a reverse osmosis unit, and passed through a semi-permeable membrane to remove impurities and produce deionized water. Anhydrous 99% pure citric acid was mixed with the water to produce 200 gallons of a 20% (wt/vol) (796 g citric acid per gallon water) solution. The 200 gallons of 20% citric acid were directed into an ion chamber containing having positive and negative electrodes, each consisting of 200 troy ounces of 999 fine silver. The positive and negative electrodes were spaced at least 2.0 mm apart, allowing the citric acid solution to pass between the two electrodes. An ion generation controller (IGC) power supply including a positive and a negative conductor was attached to the positive and negative electrodes. The IGC applied a current of 5 amps at 17 volts, pulsed every 9 seconds, with a polarity change at 1 minute intervals. Throughout the process, the electrode gap was adjusted in order to maintain the 5 amp-17 volt output. The electric current flow caused an ion current to flow between the positive and negative electrodes, producing free silver ions within the diluted citric acid solution. The silver ions reacted with the citric acid in the citric acid solution to produce the silver dihydrogen citrate solution. The 20% citric acid solution was recirculated through the ion chamber at 50 gallons per minute for 144 hours until the desired silver ion concentration was obtained. The silver dihydrogen citrate solution was then allowed to sit in order to allow any solids formed during the procedure to precipitate. The resulting product was a silver dihydrogen citrate solution having a silver ion concentration of 2410 ppm.

The silver dihydrogen solution can be stored or it can be used immediately per the following examples.

It should be understood by those skilled in the art that numerous variations in the size and/or spacing of the electrodes and numerous variations in the peak voltage and numerous variations in the timing sequence of the intermittent voltage polarity can readily be used to obtain the silver dihydrogen citrate for use in the invention.

By the foregoing method, a solution was prepared having a silver ion concentration of 2410 ppm. The 2410 ppm silver ion solution was diluted in 5% aqueous citric acid, pH 7.0 to produce a silver dihydrogen citrate stock solution (stock solution) having a silver ion concentration of 100 ppm silver.

### EXAMPLE 2

Personal Care Formulations

Various formulations of silver dihydrogen citrate are presented in the following tables 1 through 8. In each of the following tables, the X's indicate inclusion of the indicated ingredients in the indicated proportions in the separately numbered embodiments of the invention. Also in the tables, numbers followed by percent (%) signs indicate percentages, whereas numbers not followed by a percent sign indicate parts. Unless otherwise indicated, liquid proportions are calculated as volume percents (vol/vol) and solid proportions are calculated by weight/volume percents (wt/vol). In each case, q.s. indicates that water is added to make up the remaining volume of the composition.

A stock silver dihydrogen citrate solution is prepared as described in Example 1, above. In the following tables, the silver dihydrogen citrate stock solution (stock solution) is a silver dihydrogen citrate solution having a silver ion concentration of 100 ppm. The proportion of stock silver dihydrogen citrate solution set forth in the tables is thus an expression of the volume of stock solution used in relation to the volume of the final product formed. In the examples, the concentration of silver ion in the resulting solutions will be in the range of about 100 ppb to about 20 ppm. However, the person of skill in the art will recognize that a personal care composition can be prepared having higher concentrations of silver ion by choosing a stock solution having a higher concentration of silver ion therein. For example, silver ion concentrations of up to about 200 ppm may be obtained in inventive personal care compositions using a stock solution of 1,000 ppm silver ion by following the formularies set forth below. In general, stock solutions may have concentrations of about 50 to about 10,000 ppm.

In Table 1, there are set forth several embodiments of O/W systems (oil-in-water colloidal suspensions, wherein the continuous “water” phase comprises the inventive silver dihydrogen citrate and the discontinuous “oil” phase comprises one or more water-insoluble ingredients) according to the present invention.

TABLE 1
O/W systems

| Ingredients | 1 | 2 | 3 | 4 | 5 | 6 | 7 | 8 |
|---|---|---|---|---|---|---|---|---|
| Emulsifier(s) | | | | | | | | |
| Potassium Cetyl Phosphate 2%-5% | X | | | | | | | |
| Cetearyl Alcohol/Dicetyl | | X | | | | | | |
| Phosphate/ Ceteth-10 | | | | | | | | |
| Phosphate 2%-6% | | | | | | | | |
| Sodium Stearyl Phtalamate 1%-2% | | | X | | | | | |
| Cetearyl Alcohol/ | | | | X | | | | |
| Behentrimonium Methosulfate | | | | | | | | |
| 1%-5% | | | | | | | | |
| Quaternium-32 1%-5% | | | | | X | | | |
| Dimethicone copolyol/Caprylic/ | | | | | | X | | |
| Capric Triglyceride (1%-4%) | | | | | | | | |
| Steareth-2/Steareth-21 2%-5% | | | | | | | X | |
| Polyglyceryl Methyl Glucose | | | | | | | | X |
| Distearate 1%-4% | | | | | | | | |
| Lipophilic emollient/dispersant | X | X | X | X | X | X | X | X |
| oil 15%-20% | | | | | | | | |
| Fatty Alcohols and/or Waxes | X | X | X | X | X | X | X | X |
| 1%-5% | | | | | | | | |
| Thickeners (water swellable | X | X | X | X | X | X | X | X |
| thickeners) 0.5%-1.5% | | | | | | | | |
| Preservatives 0.5%-1% | X | X | X | X | X | X | X | X |
| Chelating agents (such as EDTA) | X | X | X | X | X | X | X | X |
| 0%-0.2% | | | | | | | | |
| Antioxidants 0.05%-0.2% | X | X | X | X | X | X | X | X |
| Silver Dihydrogen Citrate | X | X | X | X | X | X | X | X |
| Stock Solution (0.1%-20%) | | | | | | | | |
| Perfume oils 0.1%-0.4% | X | X | X | X | X | X | X | X |
| Water deionized Qs 100% | X | X | X | X | X | X | X | X |

In Table 2, there are set forth several embodiments of W/O systems (i.e. compositions in. which the discontinuous “water” phase comprises the inventive silver dihydrogen citrate and the continuous “oil” phase comprises one or more water-insoluble ingredients) according to the. present invention.

TABLE 2
W/O systems

| Ingredients | 1 | 2 | 3 | 4 | 5 |
|---|---|---|---|---|---|
| Emulsifiers | | | | | |
| Polyglyceryl-2 Dipolyhydroxystearate 2%-4% | X | | | | |
| PEG-30 Dipolyhydroxystearate 2%-4% | | X | | | |
| Rapeseed Oil Sorbitol Esters 1%-5% | | | X | | |
| PEG-45/Dodecyl Glycol Copolymer 1%-5% | | | | X | |
| Sorbitan Oleate/Polycerol-3 ricinoleate 1%-5% | | | | | X |
| Lipophilic emollient/dispersant oil 10%-20% | X | X | X | X | X |
| Fatty Alcohols and/or Waxes 10%-15% | X | X | X | X | X |
| Electrolytes (NaCl, MgSO₄) 0.5%-1% | X | X | X | X | X |
| Polyol phase (Propylene glycol, glycerin) 1%-8% | X | X | X | X | X |
| Preservatives 0.3%-0.8% | X | X | X | X | X |
| Perfume oils 0.1%-0.4% | X | X | X | X | X |
| Chelating agents (such as EDTA) 0%-0.2% | X | X | X | X | X |
| Antioxidants 0.05%-0.2% | X | X | X | X | X |
| Silver Dihydrogen Citrate Stock Solution | X | X | X | X | X |
| (0.1%-20%) | | | | | |
| Water deionized Qs 100% | X | X | X | X | X |

In Table 3, there are set forth several embodiments of multiple emulsion systems according to the present invention.

TABLE 3
Multiple emulsions

| Ingredients | 1 | 2 | 3 | 4 | 5 | 6 | 7 | 8 | 9 | 10 | 11 | 12 |
|---|---|---|---|---|---|---|---|---|---|---|---|---|
| Primary emulsion W1/O | | | | | | | | | | | | |
| PEG-30 Dipolyhydroxystearate | X | | | | | | | | | X | | X |
| (2%-6%) | | | | | | | | | | | | |
| Cetyl Dimethicone Copolyol | | X | | | | | | | X | | | |
| 1%-3% | | | | | | | | | | | | |
| PEG-30 Dipolyhydroxystearate/ | | | X | | | | | X | | | | |
| Steareth-2/Steareth-21 4%-6% | | | | | | | | | | | | |
| Polyglyceryl-2 Dipolyhydroxystearate | | | | X | | | X | | | | | |
| 1%-3% | | | | | | | | | | | | |
| Polyglyceryl-6 Ricinoleate 1%-3% | | | | | X | X | | | | | X | |
| Oil phase 15%-30% | | | | | | | | | | | | |
| Fatty acid esters | X | X | X | X | X | | | | | | X | X |
| Natural and synthetic Triglycerides | | | | | | X | X | X | X | X | X | X |
| Hydrocarbon oils | X | X | X | X | X | | | | | | X | X |
| Silicone oils | | | | | | X | X | X | X | X | X | X |
| Preservatives 0.3%-0.8% | X | X | X | X | X | X | X | X | X | X | X | X |
| Water Deionized Qs 100% | X | X | X | X | X | X | X | X | X | X | X | X |
| Ionic monofunctional O/W | | | | | | | | | | | | |
| emulsifiers | | | | | | | | | | | | |
| Sorbitan Stearate/Sucrose | X | | | | | | | X | | | | X |
| Cocoate 3%-7% | | | | | | | | | | | | |
| Sucrose Laurate 3%-7% | | X | | | | | X | | | | X | |
| Poloxamer 407 3%-7% | | | X | | | X | | | X | | | |
| Polyoxyethylene(20)Sorbate | | | | X | X | | | | | X | | |
| Monoleate 3%-5% | | | | | | | | | | | | |
| Primary emulsion W1/O 50% | X | X | X | X | X | X | X | X | X | X | X | X |
| Thickeners (water swellable | X | X | X | X | X | X | X | X | X | X | X | X |
| polymers) 0.3%-1% | | | | | | | | | | | | |
| Water deionized Qs 100% | X | X | X | X | X | X | X | X | X | X | X | X |
| Perfume oils 0.1%-0.4% | X | X | X | X | X | X | X | X | X | X | X | X |
| Silver Dihydrogen Citrate Stock | X | X | X | X | X | X | X | X | X | X | X | X |
| Solution (0.1%-20%) | | | | | | | | | | | | |

In Table 4, there are set forth several embodiments of oil-in-water-in-oil emulsions systems according to the present invention. In such embodiments, the “water” phase comprises the inventive silver dihydrogen citrate.

TABLE 4
O1/W/O2 emulsions

| Ingredients | 1 | 2 | 3 | 4 | 5 | 6 | 7 | 8 |
|---|---|---|---|---|---|---|---|---|
| Primary emulsion O1/W | | | | | | | | |
| PEG-60 Hydrogenated Castor Oil | X | | | X | X | | | X |
| 25% | | | | | | | | |
| Steareth-25 25% | | X | X | | | X | X | |
| Oil phase 75% | | | | | | | | |
| Fatty acid esters | X | | X | | | | | |
| Natural and synthetic Triglycerides | | X | | X | | | | |
| Hydrocarbon oils | | | | | X | | X | |
| Silicone oils | | | | | | X | | X |
| Preservatives 0.3%-0.8% | X | X | X | X | X | X | X | X |
| Water deionized Qs 100% | X | X | X | X | X | X | X | X |
| Non ionic multifunctional W/O | X | X | X | X | X | X | X | X |
| emulsifier 2%-5% | | | | | | | | |
| Waxes 1%-5% | X | X | X | X | X | X | X | X |
| Oil phase 20%-30% | X | X | X | X | X | X | X | X |
| Silicone oils | | | | | | | | |
| Primary emulsion O1/W 15% | X | X | X | X | X | X | X | X |
| Electrolytes (NaCl, MgSO₄) | X | X | X | X | X | X | X | X |
| 0.1%-0.5% | | | | | | | | |
| Water deionized Qs 100% | X | X | X | X | X | X | X | X |
| Perfume oils 0.1%-0.4% | X | X | X | X | X | X | X | X |
| Silver Dihydrogen Citrate Stock | X | X | X | X | X | X | X | X |
| Solution (0.1%-20%) | | | | | | | | |

In Table 5, there are set forth several embodiments of microemulsion systems according to the present invention.

TABLE 5
Microemulsions

| Ingredients | 1 | 2 | 3 | 4 | 5 | 6 | 7 | 8 | 9 | 10 |
|---|---|---|---|---|---|---|---|---|---|---|
| PEG-8 Caprylic/Capric Glycerides 10%-25% | X | | | X | X | | | X | X | |
| PPG-5-ceteth-20 10%-25% | | X | X | | | X | X | | | X |
| Polyglyceryl-6 Isostearate 5%-15% | X | | X | | | | | | | |
| Polyglyceryl-3 Diisostearate 5%-15% | | X | | X | | | | | | |
| Polyglyceryl-6 Dioleate 5%-15% | | | | | X | | X | | | |
| PPG-10 Cetyl Ether 5%-15% | | | | | | X | | X | | |
| Ethoxydiglycol 5%-15% | | | | | | | | | X | X |
| Oil phase 10%-80% | X | X | X | X | X | X | X | X | X | X |
| Isostearyl Benzoate | X | X | X | X | X | X | X | X | X | X |
| Isostearyl Isostearate | X | X | X | X | X | X | X | X | X | X |
| PEG-7 Glyceryl Cocoate | X | X | X | X | X | X | X | X | X | X |
| Cyclomethicone | X | X | X | X | X | X | X | X | X | X |
| Polyalcohols/Humectants 1%-10% | X | X | X | X | X | X | X | X | X | X |
| Preservatives 0.3-0.8% | X | X | X | X | X | X | X | X | X | X |
| Perfume oils 0.1%-0.4% | X | X | X | X | X | X | X | X | X | X |
| UV-absorber as described in table 1-3 | X | X | X | X | X | X | X | X | X | X |
| 0%-30% | | | | | | | | | | |
| Silver Dihydrogen Citrate Stock | X | X | X | X | X | X | X | X | X | X |
| solution (0.1%-20%) | | | | | | | | | | |
| Water Deionized Qs 100% | X | X | X | X | X | X | X | X | X | X |

In Table 6, there are set forth several embodiments of oil-in-water sprays emulsion systems according to the present invention.

TABLE 6
O/W Spray emulsions

| Ingredients | 1 | 2 | 3 | 4 | 5 | 6 |
|---|---|---|---|---|---|---|
| Alkyl Phosphates 0.1%-5% | X | | | X | X | |
| Glucosidic derivatives 0.1%-5% | | X | X | | | X |
| Solubilizants | | | | | | |
| Ethoxylated Glyceryl ethers 0.1%-1% | X | | X | | | |
| Polysorbates 0.1%-1% | | X | | X | | |
| Ethoxylated Oleyl ethers 0.1%-1% | | | | | X | X |
| PVP/VA Copolymer 1%-10% | X | | X | | X | |
| PVM/MA Copolymer 1%-10% | | X | | X | | X |
| Oil phase 5%-20% | X | X | X | X | X | X |
| Natural oils (Meadowfoam, Jojoba, | X | X | X | X | X | X |
| Macadamia . . . ) | | | | | | |
| Fatty acids esters | X | X | X | X | X | X |
| Mineral oils | X | X | X | X | X | X |
| Silicone oils | X | X | X | X | X | X |
| Alcohol 0%-50% | X | X | X | X | X | X |
| Thickeners 0.1%-0.5% | X | X | X | X | X | X |
| Polyacrylates | X | X | X | X | X | X |
| Aluminum/Magnesium Silicates | X | X | X | X | X | X |
| Gums | X | X | X | X | X | X |
| Neutralizing agents 0%-1% | X | X | X | X | X | X |
| Polyalcohols/Humectants 1%-5% | X | X | X | X | X | X |
| Chelating agents (such as EDTA) 0%-0.2% | X | X | X | X | X | X |
| Antioxidants 0.05%-0.2% | X | X | X | X | X | X |
| Water Deionized. qs 100% | X | X | X | X | X | X |
| Perfume oils 0.1%-0.5% | X | X | X | X | X | X |
| Preservatives 0.4%-1% | X | X | X | X | X | X |
| Silver Dihydrogen Citrate Stock | X | X | X | X | X | X |
| Solution (0.1%-20%) | | | | | | |

In Table 7, there are set forth several embodiments of aqueous gels according to the present invention.

TABLE 7
Aqueous Gels

| Ingredients | 1 | 2 | 3 | 4 | 5 | 6 | 7 | 8 | 9 | 10 | 11 | 12 |
|---|---|---|---|---|---|---|---|---|---|---|---|---|
| Thickeners | | | | | | | | | | | | |
| Natural Thickener 1%-5% | X | | | | | X | X | | | | | X |
| Semi-synthetic Thickener | | X | | | X | | | X | | | X | |
| 1%-5% | | | | | | | | | | | | |
| Synthetic Thickener 0.3%-1.3% | | | X | X | | | | | X | X | | |
| Neutralizing Agents 0.5%-1.5% | X | X | X | X | X | X | X | X | X | X | X | X |
| Polyols - Humectants 5%-50% | X | X | X | X | X | X | X | X | X | X | X | X |
| Polyquaternium series 1%-5% | X | X | X | | | | X | X | X | | | |
| PVM/MA Copolymer 1%-5% | | | | X | X | X | | | | X | X | X |
| Preservatives 0.5%-1% | X | X | X | X | X | X | X | X | X | X | X | X |
| Chelating Agents (as EDTA) | X | X | X | X | X | X | X | X | X | X | X | X |
| <0.1% | | | | | | | | | | | | |
| Silver Dihydrogen Citrate | X | X | X | X | X | X | X | X | X | X | X | X |
| Stock Solution (0.1%-20%) | | | | | | | | | | | | |
| Perfume oils 0.05%-0.4% | X | X | X | X | X | X | X | X | X | X | X | X |
| Ethoxylated Glyceryl ethers | X | X | X | | | | | | | | | |
| 0.1%-5% | | | | | | | | | | | | |
| Polysorbates 0.1%-5% | | | | X | X | X | | | | | | |
| Ethoxylated Oleyl ethers | | | | | | | X | X | X | X | X | X |
| 0.1%-5% | | | | | | | | | | | | |
| Water Deionized Qs 100% | X | X | X | X | X | X | X | X | X | X | X | X |

In Table 8, there are set forth several embodiments oleogels according to the present invention.

TABLE 8
Oleogels

| Ingredients | 1 | 2 | 3 | 4 | 5 | 6 | 7 | 8 | 9 | 10 |
|---|---|---|---|---|---|---|---|---|---|---|
| Hydrogenated Lecithin 1%-10% | X | | | | | | | | | X |
| Silica Dimethyl Silylate 1%-10% | | X | | | | | | | X | |
| Silica 1%-5% | | | X | | | | | X | | |
| C₂₄₋₂₈ Alkyl Dimethicone 1%-5% | | | | X | | | X | | | |
| Aluminum or Magnesium Stearate 1%-5% | | | | | X | X | | | | |
| Polyols - Humectants 5%-70% | X | X | X | X | X | X | X | X | X | X |
| Oil phase 20%-90% | | | | | | | | | | |
| Dicaprylyl Ether | X | | | | | X | | X | | |
| Phenyl Trimethicone | | X | | | | | X | | | |
| Hydrogenated Polyisobutene | | | X | | | | | | | |
| Isopropyl Isostearate | | | | X | | | | | X | |
| Oleogel basis (Mineral oil and | | | | | X | | | | | X |
| hydrogenated Butylene/Ethylene or | | | | | | | | | | |
| Ethylene/Propylene Styrene Copolymer) | | | | | | | | | | |
| Silicone wax 1%-10% | X | X | X | X | X | X | X | X | X | X |
| Dimethiconol Behenate | X | X | X | X | X | X | X | X | X | X |
| Dimethiconol Stearate | X | X | X | X | X | X | X | X | X | X |
| Perfume oils 0.1%-0.5% | X | X | X | X | X | X | X | X | X | X |
| Antioxidants 0.05%-0.2% | X | X | X | X | X | X | X | X | X | X |
| Silver Dihydrogen Citrate Stock | X | X | X | X | X | X | X | X | X | X |
| Solution (0.1%-20%) | | | | | | | | | | |

In Table 9, there are set forth several embodiments of light/dry cosmetic oils according to the present invention.

TABLE 9
Light/dry cosmetic oils

| Ingredients | 1 | 2 | 3 | 4 |
|---|---|---|---|---|
| Hydrocarbon oils 30%-70% | X | | | X |
| Fatty acid esters branched or not 10%-50% | | X | X | |
| Silicones/Siloxanes 0%-10% | X | | X | |
| Perfluorinated oils and Perfluoroethers 0%-10% | | X | | X |
| Viscosifying agents 0%-10% | X | X | X | X |
| Esters of long chain acids and alcohols 0%-2% | X | X | X | X |
| Antioxidants 0.1%-1% | X | X | X | X |
| Solubilizants/dispersing agents 0%-5% | X | X | X | X |
| Perfume oils 0.1%-0.5% | X | X | X | X |
| Silver Dihydrogen Citrate Stock Solution (0.1%-20%) | X | X | X | X |

In Table 10, there are set forth several embodiments of foaming/mousse according to the present invention.

TABLE 10
Foaming/Mousse products

| Ingredients | 1 |
|---|---|
| SD Alcohol 40 0%-8% | X |
| Propellant 8%-15% | X |
| Nonionic Emulsifier/Surfactant 0.5%-3% | X |
| Corrosion Inhibitor 0%-1% | X |
| Perfume oils 0.1%-0.5% | X |
| Preservatives 0.1%-1% | X |
| Miscellaneous 0%-1% | X |
| Silver Dihydrogen Citrate Stock Solution (0.1%-20%) | X |

In Table 11, there are set forth several embodiments of stick products according to the present invention.

TABLE 11
Stick products

| Ingredients | 1 |
|---|---|
| Waxes 15%-30% | X |
| Natural and silicone oils 20%-75% | X |
| Lanoline derivatives 5%->50% | X |
| Esters of lanolin | X |
| Acetylated lanolin | X |
| Lanolin oil | X |
| Colorants and pigments 10%-15% | X |
| Antioxidants 0.1%-0.8% | X |
| Perfume oils 0.1%-2% | X |
| Preservatives 0.1%-0.7% | X |
| Silver Dihydrogen Citrate Stock Solution (0.1%-20%) | X |

In Table 12, there are set forth several embodiments of liquid and compact according to the present invention.

TABLE 12
Liquid and Compact

| Ingredients | 1 | 2 |
|---|---|---|
| Liquid foundation | | |
| Powder phase 10%-15% | X | |
| Oil phase 30%-40%; 75% (only for anhydrous form) | X | |
| Thickener/suspending agents 1%-5% | X | |
| Film forming polymers 1%-2% | X | |
| Antioxidants 0.1%-1% | X | |
| Perfume oils 0.1%-0.5% | X | |
| Preservatives 0.1%-0.8% | X | |
| Water deionized Qs 100% | X | |
| Compact powder | | |
| Powder phase 15%-50% | | X |
| Oil phase 15%-50% | | X |
| Polyol phase 5%-15% | | X |
| Antioxidants 0.1%-1% | | X |
| Perfume oils 0.1%-0.5% | | X |
| Preservatives 0.1%-0.8% | | X |
| For the two product forms | | |
| Silver Dihydrogen Citrate Stock Solution (0.1%-20%) | X | X |

In Table 13, there are set forth several embodiments of conditions shampoos according to the present invention.

TABLE 13
Conditioning Shampoos

| Ingredients | 1 |
|---|---|
| Primary surfactants (listed previously) 5%-10% | X |
| Secondary surfactants (listed previously) 5%-15% | X |
| Foam Stabilizers (listed previously) 0%-5% | X |
| Water deionized 40%-70% | X |
| Actives 0-10% | X |
| Conditioners | x |
| Refatting agents | x |
| Moisturizing agents | x |
| Thickeners/Rheology modifiers 0%-3% | X |
| Humectants 0%-2% | X |
| PH adjusting agents 0%-1% | X |
| Preservatives 0.05%-1% | X |
| Perfume oils 0.1%-1% | X |
| Antioxidants 0.05%-0.20% | X |
| Chelating Agents (EDTA) 0%-0.2% | X |
| Opascifying agents 0%-2% | X |
| Silver Dihydrogen Citrate Stock Solution (0.1%-20%) | X |

In Table 14, there are set forth several embodiments of antimicrobial cleansing compositions according to the present invention.

TABLE 14
Antimicrobial Cleansing Compositions

| Component | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Mineral oil | 1.00 | 1.00 | 1.00 | 1.00 | — | — | — | 1.00 | 1.00 | 1.00 |
| Propylene | 1.00 | 1.00 | 1.00 | 1.00 | 1.00 | 1.00 | 1.00 | 1.00 | 1.00 | 1.00 |
| glycol | | | | | | | | | | |
| Ammonium | 0.60 | 0.60 | 0.60 | 0.60 | 0.60 | 0.60 | 0.60 | 0.60 | 0.60 | 0.60 |
| Lauryl Sulfate | | | | | | | | | | |
| Citric Acid | 4.00 | — | — | — | — | — | — | 2.50 | 2.50 | 4.00 |
| Sodium | 3.30 | — | 2.00 | — | — | — | 3.70 | 2.00 | 2.00 | 3.20 |
| Citrate | | | | | | | | | | |
| Succinic Acid | — | 4.00 | — | — | 4.00 | 4.00 | — | — | — | — |
| Sodium | — | 3.30 | 0.00 | 0.00 | 3.20 | 3.00 | — | — | — | — |
| Succinate | | | | | | | | | | |
| Malic Acid | — | — | — | 4.00 | — | — | 4.00 | — | — | — |
| Sodium | — | — | — | 3.20 | — | — | — | — | — | — |
| Malonate | | | | | | | | | | |
| Steareth 20 | 0.55 | 0.55 | 0.55 | 0.55 | — | 0.55 | — | — | 0.08 | 0.28 |
| Steareth 2 | 0.45 | 0.45 | 0.45 | 0.45 | — | 0.45 | — | 0.45 | 0.07 | 0.23 |
| Oleth 20 | — | — | — | — | — | — | — | — | 0.08 | 0.28 |
| Oleth 2 | — | — | — | — | — | — | — | — | 0.07 | 0.23 |
| Silver | 0.15 | 0.15 | 0.15 | 0.15 | 0.15 | 0.01 | 0.50 | 0.50 | 0.15 | 0.25 |
| Dihydrogen | | | | | | | | | | |
| Citrate Stock | | | | | | | | | | |
| Solution | | | | | | | | | | |
| Thymol | — | — | — | — | — | 1.00 | — | — | — | — |
| Miscellaneous | 0.36 | 0.36 | 0.36 | 0.36 | 0.36 | 0.36 | 0.36 | 0.36 | 0.36 | 0.36 |
| Water | q.s. | q.s. | q.s. | q.s. | q.s. | q.s. | q.s. | q.s. | q.s. | q.s. |
| pH | 4.0 | 4.5 | 3.9 | 3.9 | 3.9 | 3.9 | 3.9 | 3.9 | 3.9 | 3.9 |

In Table 15, there are set forth several embodiments antimicrobial cleansing compositions according to the present invention.

TABLE 15
Antimicrobial Cleansing Compositions

| Component | Ex. 11 | Ex. 12 | Ex. 13 | Ex. 14 | Ex. 15 |
|---|---|---|---|---|---|
| Mineral oil | 1.00 | 1.00 | 1.00 | 1.00 | — |
| Propylene glycol | 1.00 | 1.00 | 1.00 | 1.00 | 1.00 |
| Ammonium Lauryl | — | — | — | — | 0.60 |
| Sulfate | | | | | |
| Ammonium Laureth | — | 5.00 | — | — | — |
| Sulfate | | | | | |
| Hostapur SAS 60 (SPS) | 1.00 | — | — | — | — |
| C₁₄-C₁₆ Sodium | — | — | 2.00 | — | — |
| α-Olefin Sulfonate | | | | | |
| Sodium Lauroyl | — | — | — | 1.00 | — |
| Sarcosinate | | | | | |
| Citric Acid | 0.055 | 7.50 | — | — | — |
| Sodium Citrate | — | 4.00 | 2.00 | — | — |
| Succinic Acid | 4.00 | — | — | — | — |
| Sodium Succinate | 0.67 | — | — | — | — |
| Malonic Acid | — | — | — | 4.00 | — |
| Malic Acid | — | — | 2.50 | — | — |
| Sodium Malonate | — | — | — | 3.20 | — |
| Salicylic Acid | — | — | — | — | 0.50 |
| Steareth 20 | 0.55 | 0.55 | 0.55 | 0.55 | 0.55 |
| Steareth 2 | 0.45 | 0.45 | 0.45 | 0.45 | 0.45 |
| Silver Dihydrogen | 0.15 | 3.00 | 0.15 | 0.01 | 0.15 |
| Citrate Stock | | | | | |
| Solution | | | | | |
| Cocamidopropyl | — | — | — | 4.00 | — |
| Betaine | | | | | |
| Polyquat 10 | — | — | — | 0.40 | — |
| Miscellaneous | 0.36 | 0.36 | 0.36 | 0.36 | 0.36 |
| Water | q.s. | q.s. | q.s. | q.s. | q.s. |
| pH | 3-6 | 3-6 | 3 | 6 | 3 |

In Table 16, there are set forth several embodiments formulations according to the present invention.

TABLE 16

| | Formulation | | | | |
|---|---|---|---|---|---|
| | 1 | 2 | 3 | 4 | 5 |
| Silver Dihydrogen Citrate Stock | 0.6 | 0.6 | 0.6 | 0.6 | 0.6 |
| Solution | | | | | |
| sodium dodecylbenzenesulfonate | 6 | 6 | 6 | 6 | 6 |
| sodium lauryl sulfate | 8 | 8 | 8 | 8 | 8 |
| Pareth 45-7 (Dobanol 45-7) | 4 | 4 | 4 | 4 | 4 |
| Ethanol | 9 | 9 | 9 | 9 | 9 |
| sodium cumenesulfonate | 5 | — | 5 | 5 | — |
| soap noodles (Mettler) | 5 | 7 | 7 | 5 | 7 |
| trisodium citrate dehydrate | 2 | 2 | 2 | 2 | 2 |
| Triethanolamine | 5 | 5 | 5 | 5 | 5 |
| fluorescent whitening agents | 0.3 | 0.3 | 0.3 | 0.3 | 0.3 |
| Water to | 100 | 100 | 100 | 100 | 100 |

B. Home and Fabric Care Formulations

In Table 17 there are set forth several home and fabric care formulations according to embodiments of the present invention.

TABLE 17

| | Formulation | | | | | | | | | | |
|---|---|---|---|---|---|---|---|---|---|---|---|
| Components | 1 | 2 | 3 | 4 | 5 | 6 | 7 | 8 | 9 | 10 | 11 |
| Silver Dihydrogen | 0.9 | 0.9 | 0.9 | 0.9 | 0.9 | 0.9 | 0.9 | 0.9 | 0.9 | 0.9 | 0.9 |
| Citrate Stock | | | | | | | | | | | |
| Solution | | | | | | | | | | | |
| dodecylbenzenesulfonic | 7.5 | | | | | | | 8.5 | | | |
| acid | | | | | | | | | | | |
| Sodium | | 27 | 23.6 | 10 | 28 | | | | 20 | 24 | 6 |
| dodecylbenzenesulfonate | | | | | | | | | | | |
| sodium laureth | 17 | 10 | | | | | | | | | |
| sulfate 3 EO | | | | | | | | | | | |
| sodium lauryl | | | | | | 6 | | | | | 8 |
| sulfate | | | | | | | | | | | |
| Coconut acid | 12.5 | | | 10 | 4 | 4 | 10 | | | 10 | |
| C₁₂₋₁₃ Pareth-7 | 10 | | | | | | | 26.9 | 27.8 | 25 | 4 |
| PEG-7 C₁₃ | | | | 20 | 9 | 14.5 | 12 | 29 | 26 | | |
| oxoalcohol | | | | | | | | | | | |
| PEG-8 C₁₃₋₁₅ fatty | | | | | | | 10 | | | | |
| alcohol | | | | | | | | | | | |
| alkyl polyglucoside | | | 5 | | | 1 | 2 | | | | |
| laureth-10 | | 5 | | | | | | | | | |
| PPG | | | | 2 | 3 | 8 | | | | | |
| sodium carbonate | | | 2 | | | | | | | | |
| sodium | | | 20 | | | | | | | | |
| tripolyphosphate | | | | | | | | | | | |
| potassium | | 22 | | | | | | | | | |
| tripolyphosphate 50% | | | | | | | | | | | |
| sodium | | 25 | | | | | | | | | |
| cumenesulfonate | | | | | | | | | | | |
| 40% | | | | | | | | | | | |
| trisodium citrate | 5.5 | | | | | 2 | | | | | 2 |
| lauryltrimonium | 0.7 | | | | | | | | | | |
| chloride | | | | | | | | | | | |
| polycarboxylate | | | | 13 | 18 | 15 | 10 | 23 | 16.2 | | |
| 2-propanol | 6 | | | 7 | 3 | | 4 | 9.5 | 8 | | |
| Ethanol | 6 | | | | | | | | | | 9 |
| Glycerol | | | | | | | | | | 20 | |
| propylene glycol | | | | | | | | | | 6 | |
| NaOH | 3.2 | | | 2 | 1 | 2.3 | 1.8 | 1.1 | | 1.8 | 4 |
| fluorescent | 0.1 | 0.1 | 0.1 | 0.1 | 0.1 | 0.1 | 0.1 | | | | 0.1 |
| whitening agent | | | | | | | | | | | |
| Tinopal CBS-x | | | | | | | | | | | |
| fluorescent | | | | | | | | 0.1 | 0.1 | 0.1 | |
| whitening agent | | | | | | | | | | | |
| Tinopal CBS-CL | | | | | | | | | | | |
| Soap | | | | | | | | | | | 7 |
| water to | 100 | 100 | 100 | 100 | 100 | 100 | 100 | 100 | 100 | 100 | 100 |

In Table 18 there are set forth several further embodiments of home and fabric care compositions according to the present invention.

TABLE 18

| | formulation | | |
|---|---|---|---|
| Components | 13a | 13b | 13c |
| Silver Dihydrogen Citrate Stock Solution | 0.9 | 0.9 | 0.45 |
| sodium laureth sulfate | 1.2 | | |
| cocamidopropyl betaine | 1 | | |
| lauramine oxide | 1 | | |
| sodium Citrate | 4 | | |
| sodium carbonate | 3 | | |
| Ethanol | 3 | | |
| sodium C₁₄₋₁₇ alkyl sec. Sulfonate | | 16.6 | |
| sodium laurylsulfate | | 20 | |
| Laureth-09 | | 3 | |
| sodium cumolsulfonate | | 5 | |
| sodium chloride | | 3 | |
| Quaternium 18 and isopropyl alcohol | | | 4 |
| Pareth-25-7 | | | 0.5 |
| water to | 100 | 100 | 100 |

In Tables 19 and 20 there are set forth embodiments of liquid washing formulations of the present invention.

TABLE 19
Liquid Washing Formulation

| | Formulation | | | | |
|---|---|---|---|---|---|
| | 1 | 2 | 3 | 4 | 5 |
| Silver Dihydrogen Citrate Stock | 0.6 | 0.6 | 0.6 | 0.6 | 0.6 |
| Solution | | | | | |
| sodium dodecylbenzenesulfonate | 6 | 6 | 6 | 6 | 6 |
| sodium lauryl sulfate | 8 | 8 | 8 | 8 | 8 |
| Pareth 45-7 (Dobanol 45-7) | 4 | 4 | 4 | 4 | 4 |
| Ethanol | 9 | 9 | 9 | 9 | 9 |
| sodium cumenesulfonate | 5 | — | 5 | 5 | — |
| soap noodles (Mettler) | 5 | 7 | 7 | 5 | 7 |
| trisodium citrate dehydrate | 2 | 2 | 2 | 2 | 2 |
| Triethanolamine | 5 | 5 | 5 | 5 | 5 |
| Fluorescent whitening agents | 0.3 | 0.3 | 0.3 | 0.3 | 0.3 |
| water to | 100 | 100 | 100 | 100 | 100 |

TABLE 20
Liquid Washing Formulation

| | Formulation | | | | | | | | | | |
|---|---|---|---|---|---|---|---|---|---|---|---|
| Components | 1 | 2 | 3c | 4 | 5 | 6 | 7 | 8 | 9 | 10 | 11 |
| Silver Dihydrogen Citrate | 0.5 | 1.0 | 0.5 | 0.2 | 0.9 | 0.6 | 1.5 | 2 | 0.5 | 0.1 | 0.2 |
| Stock Solution | | | | | | | | | | | |
| dodecylbenzenesulfonic | 7.5 | | | | | | | 8.5 | | | |
| acid | | | | | | | | | | | |
| Sodium | | 27 | 23.6 | 10 | 28 | | | | 20 | 24 | 6 |
| dodecylbenzenesulfonate | | | | | | | | | | | |
| sodium laureth sulfate 3 | 17 | 10 | | | | | | | | | |
| EO | | | | | | | | | | | |
| sodium lauryl sulfate | | | | | | 6 | | | | | 8 |
| coconut acid | 12.5 | | | 10 | 4 | 4 | 10 | | | 10 | |
| C₁₂₋₁₃ Pareth-7 | 10 | | | | | | | 26.9 | 27.8 | 25 | 4 |
| PEG-7 C₁₃ oxoalcohol | | | | 20 | 9 | 14.5 | 12 | 29 | 26 | | |
| PEG-8 C₁₃₋₁₅ fatty alcohol | | | | | | | 10 | | | | |
| alkyl polyglucoside | | | 5 | | | 1 | 2 | | | | |
| laureth-10 | | 5 | | | | | | | | | |
| PPG | | | | 2 | 3 | 8 | | | | | |
| sodium carbonate | | | 2 | | | | | | | | |
| sodium tripolyphosphate | | | 20 | | | | | | | | |
| potassium | | 22 | | | | | | | | | |
| tripolyphosphate 50% | | | | | | | | | | | |
| sodium cumenesulfonate | | 25 | | | | | | | | | |
| 40% | | | | | | | | | | | |
| trisodium citrate | 5.5 | | | | | 2 | | | | | 2 |
| lauryltrimonium chloride | 0.7 | | | | | | | | | | |
| Polycarboxylate | | | | 13 | 18 | 15 | 10 | 23 | 16.2 | | |
| 2-propanol | 6 | | | 7 | 3 | | 4 | 9.5 | 8 | | |
| Ethanol | 6 | | | | | | | | | | 9 |
| Glycerol | | | | | | | | | | 20 | |
| propylene glycol | | | | | | | | | | 6 | |
| NaOH | 3.2 | | | 2 | 1 | 2.3 | 1.8 | 1.1 | | 1.8 | 4 |
| fluorescent whitening | 0.1 | 0.1 | 0.1 | 0.1 | 0.1 | 0.1 | 0.1 | | | | 0.1 |
| agent Tinopal CBS-x | | | | | | | | | | | |
| Fluorescent whitening | | | | | | | | 0.1 | 0.1 | 0.1 | |
| agent Tinopal CBS-CL | | | | | | | | | | | |
| Soap | | | | | | | | | | | 7 |
| water to | 100 | 100 | 100 | 100 | 100 | 100 | 100 | 100 | 100 | 100 | |

In Table 21 there are set forth further liquid washing formulations according to the present invention.

TABLE 21

| Liquid Washing Formulation | formulation | | |
|---|---|---|---|
| Components | 13a | 13b | 13c |
| Silver Dihydrogen Citrate Stock Solution | 0.5 | 1.0 | 0.2 |
| sodium laureth sulfate | 1.2 | | |
| cocamidopropyl betaine | 1 | | |
| lauramine oxide | 1 | | |
| sodium Citrate | 4 | | |
| sodium carbonate | 3 | | |
| Ethanol | 3 | | |
| sodium C₁₄₋₁₇ alkyl sec. Sulfonate | | 16.6 | |
| sodium laurylsulfate | | 20 | |
| Laureth-09 | | 3 | |
| sodium cumolsulfonate | | 5 | |
| sodium chloride | | 3 | |
| Quaternium 18 and iospropyl alcohol | | | 4 |
| Pareth-25-7 | | | 0.5 |
| water to | 100 | 100 | 100 |

### EXAMPLE 3

Microbiocidal Effect of Silver Dihydrogen Citrate

Silver dihydrogen citrate was tested against several varieties of bacteria and was found to be bacteriostatic. The minimum inhibitory concentration of silver (μg/ml) was determined for each strain of bacterium as summarized as set forth in the following tables.

In the Table 22, test solutions contained 100 ppm Ag⁺ in 5% citric acid solution at pH 7.0 (adjusted with NaOH).

TABLE 22

| | Minimum Inhibitory | |
|---|---|---|
| | Concentration | |
| Bacterial Strain | (MIC) (μg/ml Ag⁺) | |
| Staphylococcus aureus (ATCC 6538), | 4.0 | μg/ml |
| Escherichia coli (ATCC 10536) | 2.0 | |
| Pseudomonas aeruginosa (ATCC 15442) | 4.0 | |
| Corynebacterium xerosis (ATCC 373) | 2.0 | |

In the Table 23, test solutions contained 100 ppm Ag⁺ in water at pH 7.0 (adjusted with NaOH).

TABLE 23

| | Minimum Inhibitory |
|---|---|
| | Concentration |
| Bacterial Strain | (MIC) (μg/ml Ag⁺) |
| Corynebacterium xerosis (ATCC 373) | 3.0 |
| Corynebacterium minutissimum (ATCC 23348) | 3.0 |
| Propionibacterium acnes (ATCC 6919) | 6.0 |
| Candida albicans (ATCC 10231) | 3.0 |
| Malessezia furfur (DSM 6171) | 6.0 |
| Chaetomium globosum (ATCC 6205) | 12-24 |
| Trichophyton mentagrophytes (ATCC 9533) | 12-24 |
| Trichophyton rubrum (ATCC 10218) | 12-48 |
| Epidermophyton floccosum DSM 10709) | 12-48 |

In the Table 24, various formulations according to the invention were tested at the indicated concentrations. In the table, 2410 ppm silver refers to a stock solution of silver dihydrogen citrate solution in which the proportion of silver, by weight, is 2410 parts per million.

TABLE 24

| | | Log₁₀ | Log₁₀ |
|---|---|---|---|
| | | reduction | reduction |
| Organism | Composition tested | (5 min.) | (10 min.) |
| Escherichia coli | 10% ethanol in water | 0 | 0 |
| (ATCC 10536) | | | |
| Escherichia coli | 0.3% of 2410 ppm | 5 | 5 |
| (ATCC 10536) | silver in 10% ethanol | | |
| Escherichia coli | 0.3% of 2410 ppm | 5 | 5 |
| (ATCC 10536) | silver in 10% ethanol | | |
| Corynebacterium | 10% ethanol + emulgin + | ˜2.4 | — |
| minutissimum (ATCC | dipropylene glycol | | |
| 23348) | | | |
| Corynebacterium | 10% ethanol + emulgin + | 5 | — |
| minutissimum (ATCC | dipropylene glycol + | | |
| 23348) | 0.3% of 2410 ppm | | |
| | silver | | |
| Corynebacterium | 10% ethanol + emulgin + | 5 | — |
| minutissimum (ATCC | dipropylene glycol + | | |
| 23348) | 0.5% of 2410 ppm | | |
| | silver | | |
| Corynebacterium | 10% ethanol + | 5 | — |
| minutissimum (ATCC | 0.5% of 2410 ppm silver | | |
| 23348) | | | |
| Staphylococcus | 0.5% xanthan gum + | 5 | 5 |
| aureus (ATCC 6538) | 2410 ppm silver + SLS | | |
| Excherichia coli | SLS + 0.5% xanthan | ˜0.6 | 2.9 |
| (ATCC 10536) | gum | | |
| Excherichia coli | 0.5% xanthan gum + | 5 | 5 |
| (ATCC 10536) | 0.5% of 2410 ppm | | |
| | silver solution + SLS | | |
| Corynebacterium | 0.5% xanthan gum | 0 | 3.6 |
| minutissimum (ATCC | | | |
| 23348) | | | |
| Corynebacterium | 0.5% xanthan gum + | 4.8 | 5 |
| minutissimum (ATCC | 0.5% of 2410 ppm | | |
| 23348) | silver solution | | |

### EXAMPLE 4

Stability Assessment in Surfactants

The time-wise stability of the antimicrobial activity of silver dihydrogen citrate was measured. The compositions comprised no surfactant, anionic surfactant, non-ionic surfactant, amphoteric surfactant, or combinations of surfactants. It was found that a mixture of anion, non-ionic and amphoteric surfactants was stable and showed good preservation with silver dihydrogen citrate.

### EXAMPLE 6

Preservation Activity

Test Method:

In order to demonstrate the antimicrobial efficacy of silver dihydrogen citrate compositions, silver dihydrogen citrate compositions per Example 1 where subjected to Preservative Challenge Tests. The Preservative Challenge Tests were performed according to the European Pharmacopoeia test method 4.04/5.01.03.00 for Category 2 products (topically used products made with aqueous bases or vehicles, nonsterile nasal products, emulsions including those applied to mucous membranes.

Bacterial test organisms and veasts were cultivated on Casein Soymeal peptone agar and fungal test organisms on Sabouraud 4% glucose agar for 18-24 hours at 35° C. (bacteria), 48 hours at 25° C. (Candida) or 1 week at 25° C. (Aspergillus).

After incubation, the bacterial and veasts were harvested by washing off the surface of the agar plates with 0.9% sodium chloride. Aspergillus was harvested by washing off the agar plate surface with 0.9% sodium chloride/0.01% Tween 80.

The suspension of test microorganisms were diluted with 0.9% sodium chloride to the final test organism suspensions with a density of ˜10⁸ colony forming units.

Per test organisms, 20 g of the test product were weight in glass jars (250 ml jars with screw cups from Schott/Germany) and contaminated with 0.2 ml of the test organism suspension. The microorganisms were carefully distributed in the test product by stirring with a glass spatula.

The so-contaminated test products were stored at 20-25° C. in the dark.

Samples of 1 g material were taken immediately after contamination of the test products and 2 days, 7 days, 14 days and 28 days after contamination.

The samples were diluted in 0.9% sodium chloride and 0.1 ml aliquots ofthe dilutions were spread on agar plates by means of Drigalsky spatula. An adequate inactivator (neutralizer) of the specific antimicrobial was incorporated in the diluent used for preparation of the product dilutions and in the agar plates used for assessment of the total number of viable cells.

The agar plates were incubated for 24 hours at 35° C. (bacteria and veasts) or 3 days at 25° C. (Aspergillus) and the grown colonies were counted after the incubation phase. The colonies were counted and the number of viable cells (colony forming units) per g test product was calculated. The log reduction of the microorganisms in the product was then calculated (see tables with results of Preservation Challenge Tests below).

Test Strains:

Pseudomonas aeruginosa ATCC 9027; NCIMB 8626; CIP 82.118

Staphylococcus aureus ATCC 6538; NCTC 10788; NCIMB 9518; CIP 4.83

Candida albicans ATCC 10231; NCPF 3179; IP 48.72

Aspergillus niger ATCC 16404; IMI 149007; IP 1431.83

Silver dihydrogen citrate was tested in a variety of formulations for its antimicrobial effects. The following tables 25-27 show the results of these tests:

TABLE 25
Preservative Challenge Test/Deodorant Emulsion

| | Test organisms | | | | |
|---|---|---|---|---|---|
| | Staph. aureus | E. coli | Ps. aeruginosa | C. albicans | A. niger |
| O/W PK03-260-01 (Placebo) | | | | | |
| 2 days after contamination | <100 | <100 | <100 | 6.0 × 10E4 | 1.8 × 10E5 |
| 7 days after contamination | <100 | <100 | <100 | 1.6 × 10E4 | 3.0 × 10E5 |
| 14 days after contamination | <100 | <100 | <100 | 8.8 × 10E3 | 2.8 × 10E5 |
| 28 days after contamination | <100 | <100 | <100 | 1.4 × 10E3 | n.d. |
| O/W PK03-260-01 (0.1% Axenohl) | | | | | |
| 2 days after contamination | <100 | <100 | <100 | <100 | 1.6 × 10E5 |
| 7 days after contamination | <100 | <100 | <100 | <100 | 2.6 × 10E5 |
| 14 days after contamination | <100 | <100 | <100 | <100 | 1.2 × 10E5 |
| 28 days after contamination | <100 | <100 | <100 | <100 | n.d. |
| O/W PK03-260-01 (0.3% Axenohl) | | | | | |
| 2 days after contamination | <100 | <100 | <100 | <100 | 1.4 × 10E5 |
| 7 days after contamination | <100 | <100 | <100 | <100 | 1.2 × 10E5 |
| 14 days after contamination | <100 | <100 | <100 | <100 | 1.0 × 10E5 |
| 28 days after contamination | <100 | <100 | <100 | <100 | n.d. |

TABLE 26
Preservative Challenge Test/Deodorant Emulsion

| | Test organisms | | | | |
|---|---|---|---|---|---|
| | Staph. aureus | E. coli | Ps. aeruginosa | C. albicans | A. niger |
| PK03-262-01 (Placebo) | | | | | |
| 2 days after contamination | 2.0 × 10E4 | 1.0 × 10E5 | 1.0 × 10E6 | 1.0 × 10E6 | 2.0 × 10E5 |
| 7 days after contamination | <100 | 3.1 × 10E3 | 4.4 × 105 | 6.2 × 10E5 | 3.0 × 10E5 |
| 14 days after contamination | <100 | 1.0 × 10E2 | 1.1 × 10E6 | 1.3 × 10E6 | 3.5 × 10E5 |
| 28 days after contamination | <100 | 1.2 × 10E2 | 6.0 × 10E7 | 1.1 × 10E6 | n.d. |
| PK03-262-01 (0.1% Axenohl) | | | | | |
| 2 days after contamination | 5.0 × 10E2 | 4.0 × 10E2 | 3.0 × 10E2 | 4.0 × 10E3 | 1.4 × 10E5 |
| 7 days after contamination | <100 | 2.0 × 10E2 | <100 | 3.4 × 10E4 | 1.8 × 10E5 |
| 14 days after contamination | <100 | <100 | <100 | 1.1 × 10E5 | 2.0 × 10E5 |
| 28 days after contamination | <100 | <100 | <100 | 4.0 × 10E5 | n.d. |
| PK03-262-01 (0.3% Axenohl) | | | | | |
| 2 days after contamination | 3.0 × 10E2 | 4.0 × 10E3 | 7.0 × 10E2 | 4.0 × 10E3 | 1.8 × 10E5 |
| 7 days after contamination | <100 | 2.0 × 10E2 | <100 | 2.4 × 10E3 | 2.4 × 10E5 |
| 14 days after contamination | <100 | 100 | <100 | 1.2 × 10E3 | 2.4 × 10E5 |
| 28 days after contamination | <100 | <100 | <100 | 2.0 × 10E2 | n.d. |

TABLE 27
Preservative Challenge Test/Shower Gel

| | Test organisms | | | | |
|---|---|---|---|---|---|
| | Staph. aureus | E. coli | Ps. aeruginosa | C. albicans | A. niger |
| FB02-060-03 (Placebo) | | | | | |
| 2 days after contamination | 4.0 × 10E2 | 2.2 × 10E5 | in progress | 2.2 × 10E5 | 2.4 × 10E5 |
| 7 days after contamination | <100 | 1.1 × 10E5 | in progress | 2.1 × 10E5 | 3.6 × 10E5 |
| 14 days after contamination | <100 | 8.2 × 10E4 | in progress | 9.0 × 10E4 | 3.6 × 10E5 |
| 28 days after contamination | n.d. | n.d. | in progress | n.d. | n.d. |
| Body Shampoo (0.1% Axenohl) | | | | | |
| 2 days after contamination | <100 | 1.8 × 10E4 | <100 | <100 | 2.0 × 10E5 |
| 7 days after contamination | <100 | <100 | <100 | <100 | 3.0 × 10E5 |
| 14 days after contamination | <100 | <100 | <100 | <100 | 3.2 × 10E5 |
| 28 days after contamination | <100 | <100 | <100 | <100 | n.d. |
| Body Shampoo (0.3% Axenohl) | | | | | |
| 2 days after contamination | <100 | 6.0 × 10E3 | <100 | <100 | 1.8 × 10E5 |
| 7 days after contamination | <100 | <100 | <100 | <100 | 2.4 × 10E5 |
| 14 days after contamination | <100 | <100 | <100 | <100 | 2.4 × 10E5 |
| 28 days after contamination | <100 | <100 | <100 | <100 | n.d. |

While the invention has been described with reference to the above examples, it should be understood that one of skill in the art will recognize that other embodiments can be prepared and are within the ambit of the present invention.

The references, including all United States patent documents, cited herein are incorporated herein by reference.

What is claimed is:

1. A personal care composition, comprising silver dihydrogen citrate and a physiologically acceptable medium.

2. The composition of claim 1, further comprising at least one additional ingredient selected from the group consisting of a deodorant, an antiperspirant, an antimicrobial agent other than an alcohol, a proton donating agent, a mildness enhancing agent, a skin moisturizer, a humectant, an emollient, an oil, a lipid-type material, a stabilizer, an abrasive, an anti-acne agent, an antioxidant, a colorant, an astringent, a film former, a fragrance component, an opacifying agent, a propellant, a reducing agent, a skin bleaching agent or a sunscreen agent, and an oral care agent, or combinations of two or more thereof.

3. The composition of claim 1, further comprising one or both of an alcohol or a detergent.

4. A personal care composition, comprising silver dihydrogen citrate, water, and at least one ingredient other than a detergent or an alcohol.

5. The composition of claim 4, wherein the ingredient other than alcohol or detergent is at least one of a deodorant, an antiperspirant, an additional antimicrobial agent other than an alcohol, an additional proton donating agent, a mildness enhancing agent, a skin moisturizer, a humectant, an emollient, an oil, a lipid-type material, a stabilizer, an abrasive, an anti-acne agent, an antioxidant, a colorant, an astringent, a film former, a fragrance component, an opacifying agent, a propellant, a reducing agent, a skin bleaching agent, a sunscreen agent or combinations of two or more thereof.

6. A personal care composition, comprising silver dihydrogen citrate, water, an oil phase and at least one emulsifying agent.

7. The personal care composition of claim 6, which is a water-in-oil emulsion, an oil-in-water emulsion, an oil-in-water-in-oil emulsion or a water-in-oil-in-water emulsion,

8. The personal care composition of claim 6, which is a microemulsion, a macroemulsion or a phase inversion temperature emulsion.

9. A personal care composition, comprising a mixture of a water-insoluble solid ingredient in an aqueous phase comprising silver dihydrogen citrate and water.

10. The personal care composition of claim 9, wherein the composition is a paste or a colloidal suspension.

11. A personal care composition, comprising silver dihydrogen citrate, water and at least one member of the group consisting of gelling agents, thickening agents, and mixtures thereof.

12. The personal care composition of claim 1 1, comprising silver dihydrogen citrate, water, and a gelling agent.

13. The personal care composition of claim 12, in liquid, semi-solid or solid form.

14. The personal care composition of claim 12, further comprising one or more members of the group consisting of deodorants, antiperspirants and fragrances.

15. The personal care composition of claim 12, further comprising one or more oral care ingredients.

16. The personal care composition of claim 15, wherein the oral care ingredients comprise cleaning agents, polishing agents, fluoridating agents, malodor treating agents, tooth whitening agents, anti-carries agents, gelling agents, antibacterial agents other than silver dihydrogen citrate, flavorings, colorants and combinations of two or more of the foregoing.

17. A personal care composition, comprising an emulsion having a water phase that contains silver dihydrogen citrate and water, an oil phase, and an emulsifier, wherein the emulsion is in a form selected from the group consisting of: a water-in-oil emulsion, an oil-in-water emulsion, an oil-in-water-in-oil emulsion, a water-in-oil-in-water emulsion, a phase inversion temperature emulsion, or a microemulsion.

18. A personal care composition, comprising silver dihydrogen citrate and water in an orally acceptable form selected from the group consisting of: a mouth rinse, a mouth wash, a tooth paste, a tooth gel, a denture paste, a denture gel, a chewing gum, a solid lozenge or an oral spray.

19. A home care composition, comprising silver dihydrogen citrate, water, and at least one ingredient other than a detergent or an alcohol.

20. The home care composition of claim 19, wherein the ingredient other than detergent or alcohol is an emulsifier, a gelling agent, a thickener, an essential oil, fragrance, an abrasive, a bleach, a whitener, a deodorizer, an enzyme or a stabilizer.

21. A home care composition, comprising silver dihydrogen citrate, water, an oil phase and at least one emulsifying agent.

22. A home care composition, comprising a mixture of a water-insoluble solid ingredient in a water phase comprising water and silver dihydrogen citrate.

23. A home care composition, comprising silver dihydrogen citrate, water and a gelling or thickening agent.

24. A home care composition comprising silver dihydrogen citrate and water in a suitable form selected from liquid, semi solid and solid.

25. A home care composition comprising silver dihydrogen citrate, and further comprising one or more additional ingredients selected from the group consisting of builders, enzymes, bleaches, whiteners, color care agents, fabric softeners, suds suppressors, dispersants and dye transfer inhibitors, chelating agents and aerosol propellants.

26. An antimicrobial laundry care composition, comprising water and silver dihydrogen citrate in the form of a liquid, paste, gel, bar, tablet, spray, foam, powder or granule.

27. A method of using a personal care composition of claims 1, 4, 6, 9, 11, 17, 19, 21, 22, 23, comprising applying an antimicrobially effective amount of the personal care composition to a human body surface or part.

28. A method of using a home care composition of claim 24, 25 or 26, comprising contacting an antimicrobially effective amount of the home care composition with an article or surface.

29. A personal care composition of claim one where the composition is formulated as a deodorant, and antiperspirant, skin care product, sun screen product, personal cleansing product, hair care product, oral care product or decorative cosmetic.
